(19) United States
(12) Patent Application Publication
(10) Pub. No.: US 2023/0257468 A1
(43) Pub. Date: Aug. 17, 2023

(54) CIS-Binding Siglec Agonists and Related Compositions And Methods

(71) Applicant: The Board of Trustees of the Leland Stanford Junior University, Stanford, CA (US)

(72) Inventors: Carolyn R. Bertozzi, Menlo Park, CA (US); Corleone Delaveris, Redwood City, CA (US)

(21) Appl. No.: 18/012,199

(22) PCT Filed: Jun. 29, 2021
(86) PCT No.: PCT/US2021/039623

§ 371 Date: Dec. 21, 2022

Related U.S. Application Data
(60) Provisional application No. 63/046,140, filed on Jun. 30, 2020.

(51) Int. Cl. C07K 16/28; C07K 14/705; A61K 9/19; A61K 9/00; A61P 31/14

(52) U.S. Cl. CPC C07K 16/2851; C07K 14/7056; A61K 9/19; A61K 9/0019; A61P 31/14

(57) ABSTRACT

Provided are cis-binding Siglec agonists. In certain embodiments, the cis-binding Siglec agonists comprise a scaffold bearing Siglec ligands, and a membrane-tethering domain. Also provided are compositions, e.g., pharmaceutical compositions, comprising any of the cis-binding Siglec agonists of the present disclosure. Methods of agonizing Siglec activity, e.g., in an individual in need thereof, are also provided. Kits comprising the cis-binding Siglec agonists, as well as methods of making the cis-binding Siglec agonists, are also provided.

### CROSS-REFERENCE TO RELATED APPLICATIONS

This application claims the benefit of U.S. Provisional Patent Application No. 63/046,140, filed Jun. 30, 2020, which application is incorporated herein by reference in its entirety.

### STATEMENT OF GOVERNMENT SUPPORT

This invention was made with Government support under contract CA227942 awarded by the National Institutes of Health. The Government has certain rights in the invention.

### INTRODUCTION

Sialic acid binding IgG-like lectins (Siglecs) are a family of immune checkpoint receptors expressed on all classes of immune cells. They bind various sialoglycans on target cells and deliver signals to the immune cells that report on whether the target is healthy or damaged, “self” or “non-self”. Of the fourteen human Siglecs, nine contain cytosolic inhibitory signaling domains. Accordingly, engagement of these inhibitory Siglecs by sialoglycans suppresses the activity of the immune cell, leading to an anti-inflammatory effect. In this regard, inhibitory Siglecs have functional parallels with the T cell checkpoint receptors CTLA-4 and PD-1. As with these clinically established targets for cancer immune therapy, there has been a recent surge of interest in antagonizing Siglecs to potentiate immune cell reactivity toward cancer. Conversely, engagement of engagement of Siglecs with agonist antibodies can suppress immune cell reactivity in the context of anti-inflammatory therapy. This approach has been explored to achieve B cell suppression in lupus patients by agonism of CD22 (Siglec-2), and to deplete eosinophils for treatment of eosinophilic gastroenteritis by agonism of Siglec-8.

### SUMMARY

Provided are cis-binding Siglec agonists. In certain embodiments, the cis-binding Siglec agonists comprise a scaffold bearing Siglec ligands, and a membrane-tethering domain. Also provided are compositions, e.g., pharmaceutical compositions, comprising any of the cis-binding Siglec agonists of the present disclosure. Methods of agonizing Siglec activity, e.g., in an individual in need thereof, are also provided. Kits comprising the cis-binding Siglec agonists, as well as methods of making the cis-binding Siglec agonists, are also provided.

### BRIEF DESCRIPTION OF THE FIGURES

FIG. 1: Glycopolypeptides cluster and agonize Siglecs in cis on effector cells. (A) Phagocytes express activating receptors that engage “eat-me” signals on target cells, stimulating phagocytosis and inflammation. (B) Clustering of Siglec-9 by cis-ligands stimulates inhibitory signaling that quenches phagocyte activation.

FIG. 2: Representative synthesis of pS9L-lipid. (a) THF, 3, 6 h at 22° C., glovebox. (b) hydrazine monohydrate, MeOH/THF/H₂O, 24 h at 22° C.; 85% over two steps. (c) 4, sodium pyruvate, Pd26ST, NmCSS, NanA, 20 mM MgCl₂ in 200 mM Tris pH 8.5, 48 h; 50%. (d) benzhydrylazide, CuSO₄, BTTAA, tBuOH/H₂O, 12 h at 22° C.; 75-100%.

FIG. 3: Engineered glycopolypeptides bind Siglec-9 with high affinity. (A) Glycopolypeptides are based on the same lactosylserine scaffold. pLac bears only lactose moieties. pSia bears terminal Neu5Ac. pS9L bears Siglec-9 ligands. pS7L bears Siglec-7 ligands. The N-terminus of polypeptides was functionalized with either a fluorophore or biotin moiety. (B) Soluble glycopolypeptide bearing an N-terminal biotin was bound to streptavidin-coated tips. Association/dissociation curves were measured by dipping the tips into solutions of recombinant Siglec-Fc fusion proteins followed by buffer only. Data are representative of two independent experiments. (C) THP-1 monocytes were coated with lipid-tethered glycopolypolypeptides and stained with Siglec-9-Fc followed by an anti-human AlexaFluor647-conjugated secondary antibody. Data are representative of three independent experiments.

FIG. 4: pS9L-lipid associates in cis with Siglec-9 but not Siglec-7. (A) A FRET experiment to assess association of pS9L-lipid or pS7L-lipid with Siglec-9 or Siglec-7 in cis. Lipid-conjugated glycopolypeptide (pS9L-lipid or pS7L-lipid) was functionalized at the N-terminus with AlexaFluor555 and loaded onto JURKATs stably overexpressing either Siglec-9 or Siglec-7. Anti-Siglec antibodies bearing AlexaFluor647 were bound to Siglec and FRET signal was quantified by fluorescence microscopy. (B,D) Relative FRET efficiency was calculated when pS9L-lipid or pS7L-lipid was loaded onto Siglec-9 expressing cells. Statistical analysis by one-way t-test; p<0.001, Glass' Δ=6.70. (C,E) Relative FRET efficiency was calculated when pS9L-lipid or was loaded onto Siglec-9 or Siglec-7 expressing cells. Statistical analysis by one-way t-test; p<0.001, Glass' Δ=2.42. All data are representative of at least two independent experiments. Data points in (C) and (E) represent individual cells from a single experiment. Error bars are presented as SD.

FIG. 5: pS9L-lipid agonizes Siglec-9 to inhibit TLR4 signaling in an NF-κB transcription reporter assay. (A) HEKBlue cells coexpress an NF-κB-dependent secreted alkaline phosphatase (SEAP) and the TLR4 signaling complex. Upon stimulation with LPS, SEAP in the supernatant can be quantified using a colorometric assay as a proxy for NF-κB activity. For these assay, HEKBlue cells were also transfected with pCMV-Siglec expression vectors. (B) Siglec-9-expressing HEKBlue cells were grown on plates coated with antibody (anti-Siglec-9, isotype, or vehicle) and relative NF-κB transcription in response to LPS (10 ng/mL) was measured. (C) Siglec-9 expressing HEKBlue cells were pretreated with pS9L-sol (1 μM), pS9L-lipid (1 μM), or vehicle prior to LPS stimulation (10 ng/mL). (D) HEKBlue cells were transfected with Siglec-9, Siglec-7, or a mock expression vector and coated with pS9L-lipid (1 μM) or vehicle followed by LPS stimulation (10 ng/mL). (E) HEKBlue cells were transfected with a wild-type, R120A, or Y433/456F Siglec-9 expression vector and coated with pS9L-lipid (1 μM) or vehicle followed by LPS stimulation (10 ng/mL). Statistics were determined by one-way (B,C) or two-way (D,E) ANOVA, **=p<0.01, ***=p<0.001, ****=p<0.0001. Error bars are presented as SD. All data are representative of at least three independent experiments.

FIG. 6: Activation of macrophages is inhibited by cis-binding pS9L-lipid but not soluble trans-binding pS9L-sol. (A) Hyperinflammatory macrophages were pretreated with glycopolypeptide (500 nM) and subsequently subjected to LPS stimulation (100 pg/mL). Activation was assayed by cytokine quantitation from the supernatant (B), quantitative phosphoproteomics (C-E), or Western blot (F). (B) Macrophages were pretreated with glycopolypeptide (500 nM) and then stimulated with LPS (100 pg/mL) for 18 h. Aliquots of supernatant were analyzed by a multiplexed inflammatory cytokine assay. Data are presented as fold-change of averages of technical replicates from three independent experiments relative to vehicle-pretreated cells. Statistics were determined by multiple t-tests, *=p<0.05. Error bars are presented as SD. (C-E) Macrophages were pretreated with glycopolypeptide (500 nM) and then stimulated with LPS (100 pg/mL) for 5 min and lysed. Lysates were collected from three independent differentiations of macrophages. Lysates were normalized, enriched for phosphoproteins, labeled, and analyzed by quantitative phosphoproteomics. (C) A heatmap of fold change from macrophages pretreated with glycopolypeptides with or without LPS stimulation. (D) A volcano plot of significance vs. fold change over vehicle of macrophages pretreated with pS9L-lipid and stimulated with LPS. Significantly changed phosphopeptides identified are shown in red. Select unique hits are highlighted in dark blue. (E) As D, but for pS9L-sol. (F) Macrophages were treated with glycopolypeptide (500 nM) and stimulated with LPS (100 pg/mL) for 1 h before lysis and analysis by Western blot for total IkB and pIkB (S32/36) levels. Lane 1 shows control macrophages that were treated with neither glycopolypeptide nor LPS. FC is fold-change.

FIG. 7: pS9L-lipid inhibits macrophage phagocytosis in a Siglec-9 dependent manner. (A) Macrophage phagocytosis can be determined via fluorescence microscopy using beads that undergo fluorescence turn-on in acidic (i.e. late endosomal/lysosomal) compartments. (B) Representative images of merged phase and red fluorescence at 0 h (top) and 15 h (bottom). (C) THP-1 macrophages were pretreated with polymer (200 nM) and a suspension of 1 μm pHrodo red labeled beads was added at a given effector:target (E:T) ratio. The initial rate of phagocytosis was determined by measuring the increase in red fluorescent area over the first hour. Data are representative of three independent experiments. (D-F) CMAS KO (D), Siglec-9 KO (E), or wild-type (F) THP-1 macrophages were pretreated with glycopolypeptide (200 nM) and assayed for phagocytosis hourly for 10 h at an E:T ratio of 1:20 using 1 μm pHrodo red labeled beads. (G,H) BV2 murine microglia with either a CRISPR safe-targeting guide (G) or a Siglec-E KO (H) were pretreated with neuraminidase (2 μM) and then loaded with the Siglec-E cross-reactive pS7L-lipid (500 nM) before assaying phagocytosis as in D-F. For (D-H) Statistical analysis by two-way ANOVA: #=p<0.15; *=p<0.05; **=p<0.01. Error bars are SEM. Data are representative of three independent experiments.

FIG. 8: Response to pS9L-lipid by monocyte-derived primary macrophages is stratified by Siglec-9 expression. (A-D) Monocytes were isolated from PBMCs, differentiated into or M1 macrophages by treatment with GM-CSF (50 ng/mL) for 6 d. (A-C) M1 macrophages differentiated from PBMCs isolated from three different donors were treated with glycopolypeptide (500 nM) before assaying phagocytosis of pHrodo-labeled beads at approximately a 1:20 E:T ratio. Statistical analysis by two-way ANOVA, *=p<0.05. Error bars are presented as SEM. (D) M1 macrophages were stained by microscopy with a fluorescently-labeled anti-Siglec-9 antibody and fluorescence was quantified by microscopy. Donors A-C correspond to panels A-C. Normalized expression was determined taking the ratio of the integrated fluorescence intensity per image by the confluency per image. Error bars are presented as SD. Statistical analysis by two-way ANOVA, ****=p<0.001. Data are from three different donors.

FIG. 9: Synthetic glycopolypeptides bearing high-affinity Siglec-9 ligands engage Siglec-9 and induce clustering and signaling. (a) Membrane-anchored, cis binding glycopolypeptide 1 (pS9L) induces Siglec-9 signaling, while a soluble control polypeptide 2 (pS9L-sol) or a non-binding but membrane-anchored control polypeptide 3 (pLac) do not. (b) Structures of the polypeptides pS9L, pS9L-sol, and pLac. Polypeptides are all based on an O-lactosyl poly-serine-co-alanine scaffold, and in some cases bear terminal Siglec-9-binding sialic acid analogs and/or C-terminal membrane-anchoring lipids.

FIG. 10: A cis-binding Siglec-9 agonist (pS9L) inhibits R848-induced NETosis via Siglec-9 and SHP-1. (a-c) Primary neutrophils were cotreated with R848 (10 μM) and glycopolypeptide (500 nM) in IMDM supplemented 0.5% hiFBS containing the membrane impermeable DNA intercalators Cytotox Green or Red (250 nM). Images were acquired by fluorescence microscopy every 15 min for 12 h. The area of all green fluorescent objects >300 μm² was quantified and the total area was averaged across three images per well. Relative NETosis was determined by normalizing to the maximal NET area from R848 treatment alone (t=8 h). (a) Representative phase contrast and fluorescence images from t=8 h. Scale bars indicate 40 μm. (b) Quantitation of NETosis over time as area under the curve in (c). Error bars represent SD. (c) NET formation and degradation as a function of time. Error bands represent SEM. (d) Treatment of R848-stimulated neutrophils with various glycopolypeptides. Error bars represent SD. (e) pS9L is a mucin-like glycopolypeptide that bears high affinity and specific ligands for Siglec-9 and is functionalized with a membrane-tethering lipid tail. (f) HL-60 cells were transfected with siRNAs against SIGLEC9 (encoding Siglec-9), PTPN6 (encoding SHP-1), or a scrambled control and then grown for two days. Cells were then cotreated with R848 (10 μM) and vehicle or pS9L (500 nM). Relative NETosis is determined as in (b), except all objects >200 μm² were quantified and the R848 maximum in dHL-60's was observed at 2.5 h post induction. Error bars represent SD. Statistics were determined by two-way ANOVA (b) or one-way ANOVA (c,d,f). * p<0.05; ** p<0.01; *** p<0.001; **** p<0.0001.

FIG. 11: The Siglec-9 agonist pS9L inhibits NETosis of neutrophils induced by COVID-19 plasma. (a,b) Analysis of publicly available single-cell transcriptomics data 8 for SIGLEC9 expression (a) and PADI4 expression (b) on neutrophils in peripheral blood from healthy donors or COVID-19 patients. Error bars represent SD. Statistics were determined using mixed effects model. **=p<0.01; ***=p<0.001 (c,d) Primary neutrophils were cultured in undiluted and citrate anticoagulated plasma from healthy donors or COVID-19 patients for 4 h. Cells were fixed, stained for extracellular myeloperoxidase, and imaged in DAPI imaging media by fluorescence microscopy. Cells were treated in technical triplicate and imaged across multiple fields of view. (c) Proportion of NET-positive cells (%) across all fields of view. Each dot represents and individual plasma sample. (d) Representative images from a COVID-19 patient plasma sample with or without pS9L. Error bars represent SD. Statistics were determined using mixed effects models to account for samples using repeat neutrophil donors. ****=p<0.0001.

FIG. 12: Local and peripheral inflammatory stimuli induce NETosis and a subsequent hyperinflammatory cascade, e.g., in COVID-19. Both local inflammatory stimuli at the site of SARS-CoV-2 infection (e.g., virions) and peripheral inflammatory stimuli (e.g., the proinflammatory cytokines IL-8 and G-CSF) associated with COVID-19 have been shown to induce NETosis in vitro. These factors are suspected to be causative agents of NETosis in vivo as well, initiating a deleterious hyperinflammatory cascade leading to the symptoms of moderate and severe COVID-19. Based on the present disclosure, agonists of the neutrophil-associated checkpoint receptor Siglec-9 are expected to inhibit NETosis generally and in COVID-19 in particular.

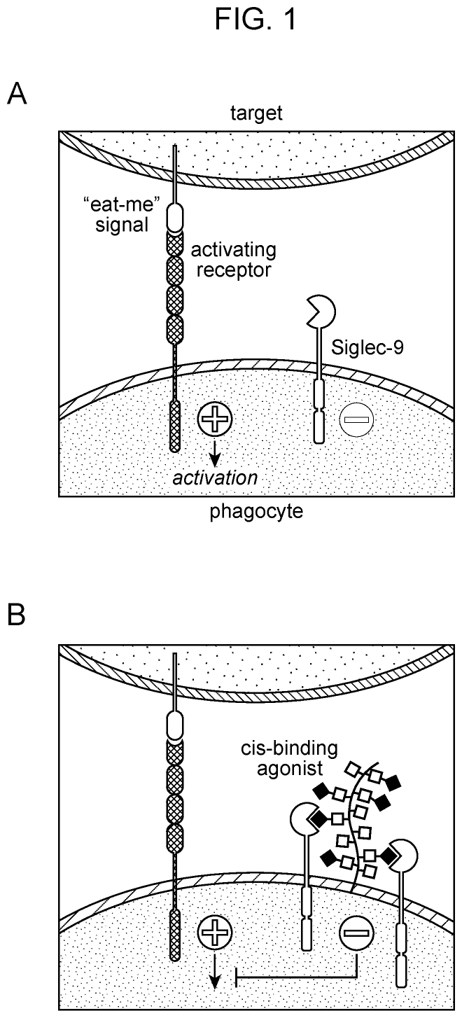

### DETAILED DESCRIPTION

Provided are cis-binding Siglec agonists. In certain embodiments, the cis-binding Siglec agonists comprise a scaffold bearing Siglec ligands, and a membrane-tethering domain. Also provided are compositions, e.g., pharmaceutical compositions, comprising any of the cis-binding Siglec agonists of the present disclosure. Methods of agonizing Siglec activity, e.g., in an individual in need thereof, are also provided. Kits comprising the cis-binding Siglec agonists, as well as methods of making the cis-binding Siglec agonists, are also provided.

Before the Siglec agonists, compositions, kits and methods of the present disclosure are described in greater detail, it is to be understood that the Siglec agonists, compositions, kits and methods are not limited to particular embodiments described, as such may, of course, vary. It is also to be understood that the terminology used herein is for the purpose of describing particular embodiments only, and is not intended to be limiting, since the scope of the Siglec agonists, compositions, kits and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the Siglec agonists, compositions, kits and methods. The upper and lower limits of these smaller ranges may independently be included in the smaller ranges and are also encompassed within the Siglec agonists, compositions, kits and methods, subject to any specifically excluded limit in the stated range. Where the stated range includes one or both of the limits, ranges excluding either or both of those included limits are also included in the Siglec agonists, compositions, kits and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the Siglec agonists, compositions, kits and methods belong. Although any Siglec agonists, compositions, kits and methods similar or equivalent to those described herein can also be used in the practice or testing of the Siglec agonists, compositions, kits and methods, representative illustrative Siglec agonists, compositions, kits and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present Siglec agonists, compositions, kits and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the Siglec agonists, compositions, kits and methods, which are, for clarity, described in the context of separate embodiments, may also be provided in combination in a single embodiment. Conversely, various features of the Siglec agonists, compositions, kits and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present Siglec agonists, compositions, kits and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Cis-Binding Siglec Agonists

As summarized above, the present disclosure provides cis-binding Siglec agonists (also referred to herein as “Siglec agonists”). According to some embodiments, the Siglec agonists comprise a scaffold bearing Siglec ligands, and a membrane-tethering domain. As demonstrated herein, the Siglec agonists spontaneously insert into cell membranes and bind specific Siglecs in cis on the surface of immune cells. The Siglec agonists find use in a variety of in vitro and in vivo applications. Unexpectedly, when tethered to the cell membrane but not as a soluble agent at comparable concentrations, the present Siglec ligands engage Siglecs and inhibit inflammatory activity in reporter systems, macrophage cell lines, and primary macrophages. As such, the Siglec agonists of the present disclosure constitute, inter alia, a novel modality of immunosuppression by engineering cis interactions into the glycocalyx. Details regarding embodiment of the Siglec agonists of the present disclosure will now be described.

The sialic acid-binding immunoglobulin-like lectins (Siglecs) are a family of immunomodulatory receptors whose functions are regulated by their glycan ligands. The Siglec family consists of 15 family members in humans that are expressed on a restricted set of cells in the hematopoietic lineage, with exceptions including Siglec-4 (MAG) on oligodendrocytes and Schwann cells and Siglec-6 on placental trophoblasts. Through their outermost N-terminal V-set domain, Siglecs recognize sialic acid-containing glycan ligands on glycoproteins and glycolipids with unique, yet overlapping, specificities. Recognition of their ligands can affect cellular signaling through immunoreceptor tyrosine-based inhibitory motifs (ITIMs) on their cytoplasmic tails. For the majority of the Siglecs, these ITIMs have the capacity of recruiting phosphatases, therefore, these members are referred to as inhibitory-type Siglecs. Exceptions include Siglec-1 and MAG, which lack such a motif, and the activatory-type Siglecs (Siglecs-14 to -16), which are associated with immunoreceptor tyrosine-based activatory motif (ITAM)-bearing adapter proteins through a positively charge amino acid in their transmembrane region.

Siglecs can be divided into two groups based on their genetic homology among mammalian species. The first group is present in all mammals and consists of Siglec-1 (Sialoadhesin), Siglec-2 (CD22), Siglec-4, and Siglec-15. The second group consists of the CD33-related Siglecs which include Siglec-3 (CD33), -5, -6, -7, -8, -9, -10, -11, -14 and -16. Monocytes, monocyte-derived macrophages, and monocyte-derived dendritic cells have largely the same Siglec profile, namely high expression of Siglec-3, -7, -9, low Siglec-10 expression and upon stimulation with IFN-α, expression of Siglec-1. In contrast, macrophages have primarily expression of Siglec-1, -3, -8, -9, -11, -15, and -16 depending on their differentiation status. Conventional dendritic cells express Siglec-3, -7, and -9, similar to monocyte-derived dendritic cells, but in addition also express low levels of Siglec-2 and Siglec-15. Plasmacytoid dendritic cells express Siglec-1 and Siglec-5. Downregulation of Siglec-7 and Siglec-9 expression on monocyte-derived dendritic cells is observed after stimulation for 48 hours with LPS, however, on monocyte-derived macrophages Siglec expression is not changed upon LPS triggering. Siglecs are also present on other immune cells, such as B cells, basophils, neutrophils, and NK cells. Further details regarding Siglecs may be found, e.g., in Angata et al. (2015) Trends Pharmacol Sci. 36(10): 645-660; Lubbers et al. (2018) Front. Immunol. 9:2807; Bochner et al. (2016) J Allergy Clin Immunol. 135(3):598-608; and Duan et al. (2020) Annu. Rev. Immunol. 38(1):365-395; the disclosures of which are incorporated herein by reference in their entireties for all purposes.

As summarized above, in certain embodiments, the Siglec agonists comprise a scaffold bearing Siglec ligands. By “scaffold” is meant a structure suitable for displaying Siglec ligands such that the Siglec ligands are capable of binding one or more corresponding Siglecs in cis. According to some embodiments, the scaffold bearing Siglec ligands comprises a polymer scaffold. As used herein, a “polymer” is a linear series of monomers connected one to the other by covalent bonds. In certain embodiments, the polymer is a polypeptide. The terms “polypeptide”, “peptide”, or “protein” are used interchangeably herein to designate a linear series of amino acid residues connected one to the other by peptide bonds between the alpha-amino and carboxy groups of adjacent residues. The amino acids may include the 20 “standard” genetically encodable amino acids, natural amino acids with biological modification of sidechains, non-natural amino acids, or a combination thereof. According to some embodiments, the scaffold bearing Siglec ligands comprises a glycopolypeptide scaffold. Non-limiting examples of suitable glycopolypeptide scaffolds include those described in the Experimental section below.

The Siglec agonists of the present disclosure may include any suitable number of Siglec ligands. In certain embodiments, a Siglec agonist of the present disclosure comprises from 2 to 200 Siglec ligands, e.g., from 2 to 150, from 2 to 100, from 2 to 75, from 2 to 50, from 2 to 25, or from 2 to 10 Siglec ligands, such as from 4 to 8 (e.g., 6) Siglec ligands. A Siglec agonist of the present disclosure may include a single type of Siglec ligand. In other embodiments, a Siglec agonist includes two or more different types of Siglec ligands, e.g., different types of Siglec ligands for binding to the same Siglec or two or more different Siglecs.

According to some embodiments, the Siglec ligands comprise ligands for a particular Siglec. In certain such embodiments, the Siglec ligands exclusively comprise ligands for the particular Siglec. By “exclusively” in this context is meant the Siglec ligands comprise only ligands for a particular Siglec, which ligands include, but are not limited to, those selective or specific for the particular Siglec. By “selective” is meant the ligand preferentially binds to a particular Siglec as compared to its binding to one or more other Siglecs (e.g., every other Siglec), e.g., in a sample and/or in vivo. In certain embodiments, a Siglec ligand is “specific” for a particular Siglec if it binds to or associates with the Siglec with an affinity or Ka (that is, an association rate constant of a particular binding interaction with units of 1/M) of, for example, greater than or equal to about 104 M⁻¹. Alternatively, affinity may be defined as an equilibrium dissociation constant (KD) of a particular binding interaction with units of M (e.g., 10⁻⁵ M to 10⁻¹³ M, or less). In certain embodiments, specific binding means the Siglec ligand binds to the particular Siglec with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of a Siglec ligand for a Siglec can be readily determined using conventional techniques, e.g., by Bio-Layer Interferometry (BLI) (e.g., using an Octet RED96 device from ForteBio), competitive ELISA (enzyme-linked immunosorbent assay), equilibrium dialysis, by using surface plasmon resonance (SPR) technology (e.g., the BIAcore 2000 or BIAcore T200 instrument, using general procedures outlined by the manufacturer); by radioimmunoassay; or the like.

The Siglec agonists of the present disclosure may comprise immunosuppressive Siglec ligands. As used herein, an “immunosuppressive Siglec ligand” is a ligand for a Siglec that comprises a cytosolic inhibitory signaling domain, where engagement of the Siglec by a ligand for the Siglec suppresses the activity of an immune cell expressing the Siglec, e.g., leading to an anti-inflammatory effect. In certain embodiments, a Siglec agonist of the present disclosure may comprise immunosuppressive Siglec ligands, where the immunosuppressive Siglec ligands comprise ligands for a CD33-related Siglec. Examples of CD33-related Siglecs include Siglec-3 (CD33), Siglec-5, Siglec-6, Siglec-7, Siglec-8, Siglec-9, Siglec-10, Siglec-11, Siglec-14 and Siglec-16. According to some embodiments, the Siglec ligands comprise Siglec-3 ligands. In certain such embodiments, the Siglec ligands exclusively comprise Siglec-3 ligands. According to some embodiments, the Siglec ligands comprise Siglec-5 ligands. In certain such embodiments, the Siglec ligands exclusively comprise Siglec-5 ligands. According to some embodiments, the Siglec ligands comprise Siglec-6 ligands. In certain such embodiments, the Siglec ligands exclusively comprise Siglec-6 ligands. According to some embodiments, the Siglec ligands comprise Siglec-7 ligands. In certain such embodiments, the Siglec ligands exclusively comprise Siglec-7 ligands. According to some embodiments, the Siglec ligands comprise Siglec-8 ligands. In certain such embodiments, the Siglec ligands exclusively comprise Siglec-8 ligands. According to some embodiments, the Siglec ligands comprise Siglec-9 ligands. In certain such embodiments, the Siglec ligands exclusively comprise Siglec-9 ligands. According to some embodiments, the Siglec ligands comprise Siglec-10 ligands. In certain such embodiments, the Siglec ligands exclusively comprise Siglec-10 ligands. According to some embodiments, the Siglec ligands comprise Siglec-11 ligands. In certain such embodiments, the Siglec ligands exclusively comprise Siglec-11 ligands. According to some embodiments, the Siglec ligands comprise Siglec-14 ligands. In certain such embodiments, the Siglec ligands exclusively comprise Siglec-14 ligands. According to some embodiments, the Siglec ligands comprise Siglec-16 ligands. In certain such embodiments, the Siglec ligands exclusively comprise Siglec-16 ligands. In certain embodiments, a Siglec agonist of the present disclosure may comprise immunosuppressive Siglec ligands, where the immunosuppressive Siglec ligands comprise ligands for Siglec-2 (CD22). In certain such embodiments, the Siglec ligands exclusively comprise Siglec-2 ligands.

Siglec ligands (e.g., sialosides and/or analogues thereof) for binding to one or more Siglecs of interest that may be employed in the Siglec agonists of the present disclosure are known and include those described in, e.g., Courtney et al. (2009) Proc. Natl. Acad. Sci. 106(8):2500-2505; Spence et al. (2015) Sci. Transl. Med. 7(303):1-13; Perdicchio et al. (2016) Proc. Natl. Acad. Sci. 113(12):3329-3334; Shahraz et al. (2015) Sci. Rep. 5:1-17; Nycholat et al. (2019) J. Am. Chem. Soc. 141(36):14032-14037; and Rillahan et al. (2012) Angew. Chemie—Int. Ed. 51(44):11014-11018; the disclosures of which are incorporated herein by reference in their entireties for all purposes.

As summarized above, the Siglec agonists of the present disclosure include a membrane-tethering domain. By “membrane-tethering domain” is meant a domain (e.g., moiety) capable of stably associating with the cell membrane of a cell (e.g., immune cell) that expresses on its surface the Siglec to be agonized in cis by the Siglec agonist. In certain embodiments, “stably associating” means a physical association between two entities in which the mean half-life of association is one day or more in PBS at 4° C. In some embodiments, the physical association between the two entities has a mean half-life of one day or more, one week or more, one month or more, including six months or more, e.g., 1 year or more, in PBS at 4° C. According to some embodiments, the stable association arises from a covalent bond between the two entities, a non-covalent bond between the two entities (e.g., an ionic or metallic bond), or other forms of chemical attraction, such as hydrogen bonding, Van der Waals forces, and the like.

Suitable membrane-tethering domains include, but are not limited to, moieties adapted to insert into the plasma membrane of the cell. The fundamental structure of the plasma membrane is the phospholipid bilayer, which forms a stable barrier between two aqueous compartments. The plasma membranes of animal cells contain four major phospholipids (phosphatidylcholine, phosphatidylethanolamine, phosphatidylserine, and sphingomyelin), which together account for more than half of the lipid in most membranes. These phospholipids are asymmetrically distributed between the two halves of the membrane bilayer. The outer leaflet of the plasma membrane consists mainly of phosphatidylcholine and sphingomyelin, whereas phosphatidylethanolamine and phosphatidylserine are the predominant phospholipids of the inner leaflet. A fifth phospholipid, phosphatidylinositol, is also localized to the inner half of the plasma membrane. Although phosphatidylinositol is a quantitatively minor membrane component, it plays an important role in cell signaling. The head groups of both phosphatidylserine and phosphatidylinositol are negatively charged, so their predominance in the inner leaflet results in a net negative charge on the cytosolic face of the plasma membrane. In certain embodiments, the membrane-tethering domain is a homodimeric coiled-coil protein domain or a multisubunit tethering complex (MTC), including but not limited to those described in Zhi et al. (2014) F1000Prime Rep. 6:74. According to some embodiments, the membrane-tethering domain comprises a lipid membrane-tethering domain. Non-limiting examples of lipid membrane-tethering domains include those employed in the Experimental section below.

Suitable membrane-tethering domains also include moieties adapted to stably bind to the cell membrane, including any constituents thereof (e.g., membrane-associated proteins, such as transmembrane proteins). In certain embodiments, such a moiety comprises a small molecule. By “small molecule” is meant a compound having a molecular weight of 1000 atomic mass units (amu) or less. According to some embodiments, the small molecule is 750 amu or less, 500 amu or less, 400 amu or less, 300 amu or less, or 200 amu or less. In certain embodiments, the small molecule is not made of repeating molecular units such as are present in a polymer.

According to some embodiments, the moiety adapted to stably bind to the cell membrane (including any constituents thereof) is an antibody. The terms “antibody” and “immunoglobulin” include antibodies or immunoglobulins of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to the cell surface molecule of the target cell, including, but not limited to single chain Fv (scFv), Fab, (Fab′)₂, (scFv′)₂, and diabodies; chimeric antibodies; monoclonal antibodies, human antibodies, humanized antibodies (e.g., humanized whole antibodies, humanized half antibodies, or humanized antibody fragments); and fusion proteins comprising an antigen-binding portion of an antibody and a non-antibody protein.

In certain embodiments, the moiety adapted to stably bind to the cell membrane is a ligand for a cell surface molecule (e.g., a cell surface receptor) expressed on the surface of the cell. The ligand may be a circulating factor, a secreted factor, a cytokine, a growth factor, a hormone, a peptide, a polypeptide, a small molecule, a nucleic acid, or the like, that forms a complex with the cell surface molecule on the surface of the cell. In some embodiments, when the moiety is a ligand, the ligand is modified in such a way that complex formation with the cell surface molecule occurs, but the normal biological result of such complex formation does not occur. In certain embodiments, the ligand is the ligand of a cell surface receptor present on the target cell. Cell surface receptors of interest include, but are not limited to, receptor tyrosine kinases (RTKs), non-receptor tyrosine kinases (non-RTKs), growth factor receptors, cytokine receptors, etc.

In some embodiments, the moiety adapted to stably bind to the cell membrane (including any constituents thereof) is an aptamer. By “aptamer” is meant a nucleic acid (e.g., an oligonucleotide) that has a specific binding affinity for the cell surface molecule. Aptamers exhibit certain desirable properties for targeted delivery of the Siglec agonists, such as ease of selection and synthesis, high binding affinity and specificity, low immunogenicity, and versatile synthetic accessibility. Aptamers that find use in the Siglec agonists of the present disclosure include those described in Zhu et al. (2015) ChemMedChem 10(1):39-45; Sun et al. (2014) Mol. Ther. Nucleic Acids 3:e182; and Zhang et al. (2011) Curr. Med. Chem. 18(27):4185-4194.

According to certain embodiments, the moiety adapted to stably bind to the cell membrane (including any constituents thereof) is a nanoparticle. As used herein, a “nanoparticle” is a particle having at least one dimension in the range of from 1 nm to 1000 nm, from 20 nm to 750 nm, from 50 nm to 500 nm, including 100 nm to 300 nm, e.g., 120-200 nm. The nanoparticle may have any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. In certain aspects, the nanoparticle includes on its surface one or more of the other moieties described herein, e.g., antibodies, ligands, aptamers, small molecules, etc. Nanoparticles that find use in the Siglec agonists of the present disclosure include those described in Wang et al. (2010) Pharmacol. Res. 62(2):90-99; Rao et al. (2015) ACS Nano 9(6):5725-5740; and Byrne et al. (2008) Adv. Drug Deliv. Rev. 60(15):1615-1626.

According to some embodiments, a Siglec agonist of the present disclosure comprises a polymer scaffold, a Siglec ligand, a membrane-tethering domain, or any combination thereof, independently selected from those of any of the cis-binding Siglec agonists described in the Experimental section below.

A Siglec agonist of the present disclosure may be detectably labeled, e.g., with an in vivo imaging agent, a radioisotope, an enzyme which generates a detectable product, a fluorescent protein, and/or the like. The Siglec agonists may be conjugated to other moieties, such as members of specific binding pairs, e.g., biotin (member of biotin-avidin specific binding pair), and the like.

Also provided are methods of making a cis-binding Siglec agonist, e.g., any of the Siglec agonists of the present disclosure. In certain embodiments, such methods include synthesizing a polymer scaffold comprising a membrane-tethering domain at a terminus thereof, and attaching Siglec ligands to subunits of the polymer scaffold. According to some embodiments, the attaching comprises sialylating subunits of the polymer scaffold. A variety of suitable approaches are available for synthesizing polymer scaffolds and attaching Siglec ligands to subunits of such polymer scaffolds. Non-limiting examples of such approaches include those employed in the Experimental section below.

### Compositions

The present disclosure also provides compositions comprising one or any combination of the cis-binding Siglec agonists of the present disclosure.

In certain aspects, a composition of the present disclosure comprises a cis-binding Siglec agonist of the present disclosure present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino) ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Aspects of the present disclosure further include pharmaceutical compositions. In some embodiments, a pharmaceutical composition of the present disclosure includes a cis-binding Siglec agonist of the present disclosure, and a pharmaceutically acceptable carrier.

The Siglec agonists of the present disclosure can be incorporated into a variety of formulations for therapeutic administration. More particularly, the Siglec agonists can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the Siglec agonists for administration to an individual (e.g., suitable for human administration) are generally sterile and may further be free of detectable pyrogens or other contaminants contraindicated for administration to a patient according to a selected route of administration.

In pharmaceutical dosage forms, the Siglec agonists can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the Siglec agonists can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The Siglec agonists can be formulated for parenteral (e.g., intravenous, intra-arterial, intraosseous, intramuscular, intracerebral, intracerebroventricular, intrathecal, subcutaneous, etc.) administration. In certain aspects, the Siglec agonists are formulated for injection by dissolving, suspending or emulsifying the Siglec agonists in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include a Siglec agonist may be prepared by mixing the Siglec agonist having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the Siglec agonists may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the Siglec agonist against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included, e.g., in an amount of about 10 mM to 500 nM.

In some embodiments, the pharmaceutical composition includes the Siglec agonist and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Methods

The present disclosure also provides methods of using the cis-binding Siglec agonists of the present disclosure. In certain embodiments, provided are methods of agonizing Siglec activity, the methods comprising contacting a cell expressing Siglecs with any of the Siglec agonists of the present disclosure under conditions in which the membrane-tethering domain inserts into the cell membrane and the Siglec ligands bind in cis to one or more Siglecs expressed by the cell. By way of example, the method may be a method of agonizing Siglec-9 activity, wherein the Siglec agonist comprises Siglec-9 ligands. According to some embodiments, the methods of the present disclosure are performed in vitro.

In certain embodiments, the methods are performed in vivo. For example, provided are methods of agonizing Siglec activity in an individual in need thereof, the method comprising administering to the individual an effective amount of any of the Siglec agonists of the present disclosure. By “effective amount” or “therapeutically effective amount” is meant a dosage sufficient to produce a desired result, e.g., an amount sufficient to effect beneficial or desired therapeutic (including preventative) results, such as a reduction in a symptom resulting from immune cell (e.g., macrophage) activity, as compared to a control. An effective amount can be administered in one or more administrations.

According to some embodiments, the individual is in need of suppression of immune cell reactivity and the Siglec ligands comprise immunosuppressive Siglec ligands, e.g., one or more of any of the immunosuppressive Siglec ligands described elsewhere herein, e.g., ligands for one or more CD33-related Siglecs (e.g., Siglec-9), ligands for Siglec-2, or any combination thereof.

In certain embodiments, the individual has an inflammatory disease and the Siglec agonist is administered to the individual in an amount effective to treat the inflammatory disease. By “treat” or “treatment” is meant at least an amelioration of one or more symptoms associated with the inflammatory disease of the individual, where amelioration is used in a broad sense to refer to at least a reduction in the magnitude of a parameter, e.g. symptom, associated with the inflammatory disease being treated. As such, treatment also includes situations where the inflammatory disease, or at least one or more symptoms associated therewith, are completely inhibited, e.g., prevented from happening, or stopped, e.g., terminated, such that the individual no longer suffers from the inflammatory disease, or at least the symptoms that characterize the inflammatory disease.

Non-limiting examples of inflammatory diseases which may be treated according to the subject methods include age related macular degeneration, neutrophilic acute respiratory distress syndrome, systemic lupus erythematosus (SLE), eosinophilic gastroenteritis, allergy, asthma, autoimmune disease, coeliac disease, glomerulonephritis, hepatitis, inflammatory bowel disease, preperfusion injury, transplant rejection, and any combination thereof.

The pharmaceutical compositions may be administered to any of a variety of individuals. In certain aspects, the individual is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human. In certain aspects, the individual is an animal model (e.g., a mouse model, a primate model, or the like) of a condition characterized by immune cell reactivity, e.g., an inflammatory disease.

In some embodiments, a therapeutically effective amount of the cis-binding Siglec agonist (e.g., present in pharmaceutical composition comprising same) is an amount that, when administered alone (e.g., in monotherapy) or in combination (e.g., in combination therapy) with one or more additional therapeutic agents, in one or more doses, is effective to reduce the symptoms of a condition characterized by immune cell reactivity (e.g., an inflammatory disease) in the individual by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the Siglec agonist.

Dosing is dependent on severity and responsiveness of the condition characterized by immune cell reactivity (e.g., an inflammatory disease) to be treated. Optimal dosing schedules can be calculated from measurements of Siglec agonist accumulation in the body of the individual. The administering physician can determine optimum dosages, dosing methodologies and repetition rates. Optimum dosages may vary depending on the relative potency of the individual Siglec agonist and can generally be estimated based on EC₅₀s found to be effective in in vitro and in vivo animal models, etc. In general, dosage is from 0.01 μg to 100 g per kg of body weight, and may be given once or more daily, weekly, monthly or yearly. The treating physician can estimate repetition rates for dosing based on measured residence times and concentrations of the Siglec agonist in bodily fluids or tissues. Following successful treatment, it may be desirable to have the individual undergo maintenance therapy to prevent the recurrence of the disease state, where the Siglec agonist is administered in maintenance doses, ranging from 0.01 μg to 100 g per kg of body weight, once or more daily, to once every several months, once every six months, once every year, or at any other suitable frequency.

The therapeutic methods of the present disclosure may include administering a single type of Siglec agonist to the individual, or may include administering two or more types of Siglec agonists by separate administration or administration of a cocktail of different Siglec agonists.

A Siglec agonist of the present disclosure may be administered to an individual using any available method and route suitable for drug delivery, including in vivo and ex vivo methods, as well as systemic and localized routes of administration. Conventional and pharmaceutically acceptable routes of administration include intranasal, intramuscular, intra-tracheal, subcutaneous, intradermal, topical application, ocular, intravenous, intra-arterial, oral, and other enteral and parenteral routes of administration. Routes of administration may be combined, if desired, or adjusted depending upon the particular Siglec agonist and/or the desired effect. The Siglec agonist may be administered in a single dose or in multiple doses. In some embodiments, the Siglec agonist is administered parenterally, e.g., intravenously, intraarterially, or the like. In some embodiments, the Siglec agonist is administered by injection, e.g., for systemic delivery (e.g., intravenous infusion) or to a local site, e.g., a local site of inflammation.

### Kits

As summarized above, the present disclosure also provides kits. The kits find use, e.g., in practicing the methods of the present disclosure. According to some embodiments, a subject kit includes any of the pharmaceutical compositions of the present disclosure, and instructions for administering an effective amount of the pharmaceutical composition to an individual in need thereof. According to some embodiments, a kit of the present disclosure comprises a pharmaceutical composition comprising a cis-binding Siglec agonist comprising immunosuppressive Siglec ligands. Such a kit may comprise instructions for administering an effective amount of the pharmaceutical composition to an individual in need of suppression of immune cell reactivity. Such a kit may comprise instructions for administering an effective amount of the pharmaceutical composition to an individual having an inflammatory disease, non-limiting examples of which include age related macular degeneration, neutrophilic acute respiratory distress syndrome, systemic lupus erythematosus (SLE), eosinophilic gastroenteritis, allergy, asthma, autoimmune disease, coeliac disease, glomerulonephritis, hepatitis, inflammatory bowel disease, preperfusion injury, transplant rejection, and any combination thereof.

The subject kits may include a quantity of the compositions, present in unit dosages, e.g., ampoules, or a multi-dosage format. As such, in certain embodiments, the kits may include one or more (e.g., two or more) unit dosages (e.g., ampoules) of a composition that includes a Siglec agonist of the present disclosure. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular Siglec agonist employed, the effect to be achieved, and the pharmacodynamics associated with the Siglec agonist, in the subject. In yet other embodiments, the kits may include a single multi dosage amount of the composition.

Components of the kits may be present in separate containers, or multiple components may be present in a single container. A suitable container includes a single tube (e.g., vial), ampoule, one or more wells of a plate (e.g., a 96-well plate, a 384-well plate, etc.), or the like.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments.

1. A cis-binding Siglec agonist comprising:
a scaffold bearing Siglec ligands; and
a membrane-tethering domain.

2. The Siglec agonist of embodiment 1, wherein the scaffold bearing Siglec ligands comprises a polymer scaffold.

3. The Siglec agonist of embodiment 2, wherein the scaffold bearing Siglec ligands comprises a glycopolypeptide scaffold.

4. The Siglec agonist of any one of embodiments 1 to 3, wherein the scaffold comprises from 2 to 50 Siglec ligands.

5. The Siglec agonist of embodiment 4, wherein the scaffold comprises from 2 to 10 Siglec ligands.

6. The Siglec agonist of any one of embodiments 1 to 5, wherein the Siglec ligands comprise immunosuppressive Siglec ligands.

7. The Siglec agonist of embodiment 6, wherein the Siglec ligands comprise ligands for one or more CD33-related Siglecs.

8. The Siglec agonist of embodiment 7, wherein the Siglec ligands comprise Siglec-9 ligands.

9. The Siglec agonist of embodiment 8, wherein the Siglec ligands exclusively comprise Siglec-9 ligands.

10. The Siglec agonist of embodiment 7, wherein the Siglec ligands comprise Siglec-7 ligands.

11. The Siglec agonist of embodiment 10, wherein the Siglec ligands exclusively comprise Siglec-7 ligands.

12. The Siglec agonist of any one of embodiments 1 to 5, wherein the membrane-tethering domain comprises a lipid membrane-tethering domain.

13. A composition comprising the Siglec agonist of any one of embodiments 1 to 12 present in a liquid medium.

14. A composition comprising the Siglec agonist of any one of embodiments 1 to 12 present in lyophilized form.

15. A pharmaceutical composition comprising:
the Siglec agonist of any one of embodiments 1 to 12; and
a pharmaceutically acceptable carrier.

16. The pharmaceutical composition of embodiment 15, wherein the composition is formulated for parenteral administration.

17. The pharmaceutical composition of embodiment 16, wherein the composition is formulated for intravenous administration.

18. A method of agonizing Siglec activity, the method comprising contacting a cell expressing Siglecs with the Siglec agonist of any one of embodiments 1 to 12 under conditions in which the membrane-tethering domain inserts into the cell membrane and the Siglec ligands bind in cis to one or more Siglecs expressed by the cell.

19. The method according to embodiment 18, wherein the method is performed in vitro.

20. The method according to embodiment 18, wherein the method is performed in vivo.

21. The method according to any one of embodiments 18 to 20, which is a method of agonizing Siglec-9 activity, wherein the Siglec agonist comprises Siglec-9 ligands.

22. A method of agonizing Siglec activity in an individual in need thereof, the method comprising administering to the individual an effective amount of the Siglec agonist of any one of embodiments 1 to 12.

23. The method according to embodiment 22, wherein the individual is in need of suppression of immune cell reactivity and the Siglec ligands comprise immunosuppressive Siglec ligands.

24. The method according to embodiment 22 or embodiment 23, wherein the individual has an inflammatory disease and the Siglec agonist is administered to the individual in an amount effective to treat the inflammatory disease.

25. The method according to embodiment 24, wherein the individual has an inflammatory disease selected from the group consisting of: age related macular degeneration, neutrophilic acute respiratory distress syndrome, systemic lupus erythematosus (SLE), eosinophilic gastroenteritis, allergy, asthma, autoimmune disease, coeliac disease, glomerulonephritis, hepatitis, inflammatory bowel disease, preperfusion injury, transplant rejection, and any combination thereof.

26. The method according to embodiment 22 or embodiment 23, wherein the individual has a viral infection.

27. The method according to embodiment 26, wherein the viral infection is a coronavirus infection.

28. The method according to embodiment 27, wherein the coronavirus infection is a SARS-CoV-2 infection.

29. The method according to any one of embodiments 22 to 28, wherein the Siglec agonist inhibits neutrophil activation in the individual.

30. The method according to any one of embodiments 22 to 29, wherein the Siglec agonist inhibits NETosis in the individual.

31. The method according to any one of embodiments 22 to 30, wherein the Siglec ligands comprise ligands for one or more CD33-related Siglecs.

32. The method according to embodiment 31, wherein the Siglec ligands comprise Siglec-9 ligands.

33. The method according to embodiment 32, wherein the Siglec ligands exclusively comprise Siglec-9 ligands.

34. The method according to embodiment 31, wherein the Siglec ligands comprise Siglec-7 ligands.

35. The method according to embodiment 34, wherein the Siglec ligands exclusively comprise Siglec-7 ligands.

36. A kit, comprising:
the pharmaceutical composition of any one of embodiments 15 to 17; and
instructions for administering an effective amount of the pharmaceutical composition to an individual in need thereof.

37. The kit according to embodiment 36, wherein the Siglec ligands comprise immunosuppressive Siglec ligands.

38. The kit according to embodiment 37, wherein the instructions are for administering an effective amount of the pharmaceutical composition to an individual in need of suppression of immune cell reactivity.

39. A method of making a cis-binding Siglec agonist, comprising:
synthesizing a polymer scaffold comprising a membrane-tethering domain at a terminus thereof; and
attaching Siglec ligands to subunits of the polymer scaffold.

40. The method according to embodiment 39, wherein the attaching comprises sialylating subunits of the polymer scaffold.

The following examples are offered by way of illustration and not by way of limitation.

### EXPERIMENTAL

### Example 1—Glycopolypeptide Synthesis by N-Carboxyanhydride Polymerization

The design of biomimetic cis ligands for Siglecs was inspired by mucins—heavily glycosylated, polypeptides that are native Siglec ligands. To construct the glycopolypeptide backbone, a N-carboxyanhydride (NCA) polymerization platform was employed. NCA monomers were polymerized using lipid-tethered initiator to afford lipid-tethered polypeptides that spontaneously insert into cell membranes. In order to elaborate the glycopolypeptide scaffold, we combined the enzymatic methods from Chen and coworkers (Angew. Chemie—Int. Ed. 2006, 45 (24), 3938-3944) used previously (Membrane-Tethered Mucin-like Polypeptides Sterically Inhibit Binding and Slow Fusion Kinetics of Influenza A Virus. ChemRxiv 2019) with the sialic acid analogs previously reported by Paulson and coworkers to bind discrete Siglecs with high affinity and selectivity (Angew. Chemie—Int. Ed. 2012, 51 (44), 11014-11018). Assessed herein was whether such lipid-linked sialo-glycopolypeptides would insert into cell membranes and cluster neighboring Siglec receptors vis cis binding.

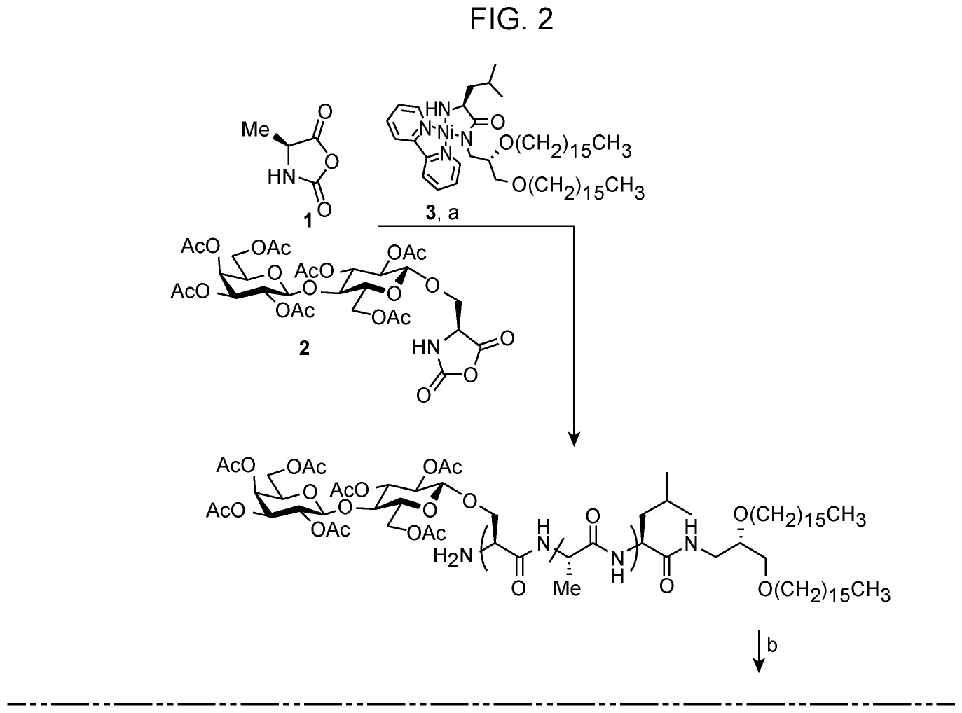

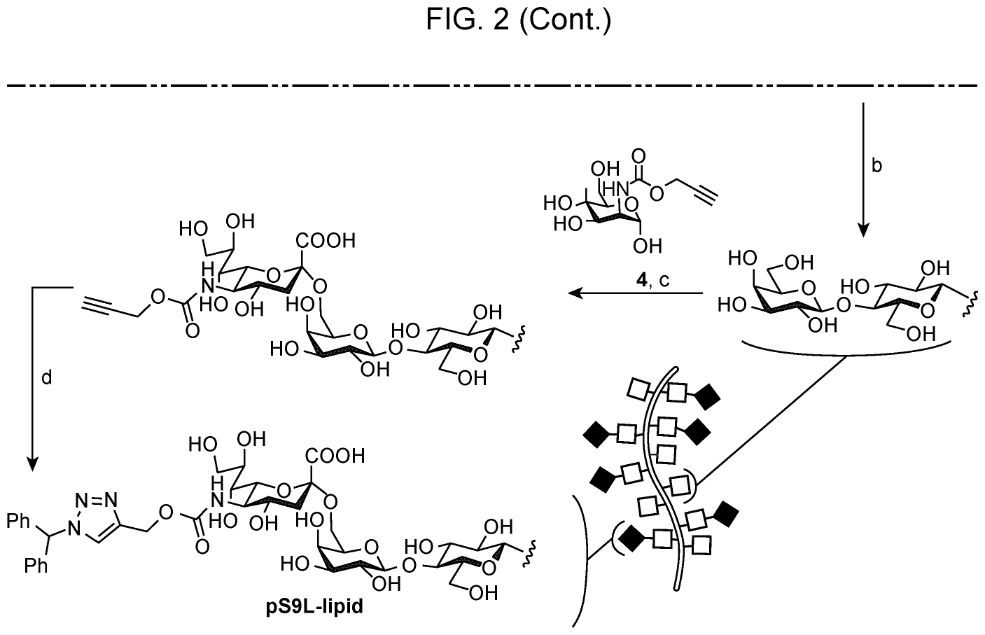

Glycopolypeptide scaffolds were synthesized by polymerization of an equimolar mixture of alanine NCA 1 and O-β-peracetyllactose serine NCA 2 (FIG. 2). Polymerizations were either initiated with a Ni(0) complex to afford a soluble glycopolypeptide or by precomplexing Ni(0) with a lipid-conjugated N-allylcarboxy leucine amide to form an activated Ni(II) initiator complex 3. The lipid-conjugated initiator affords a C-terminally conjugated lipid on the polypeptide. After polymerization, the carbohydrate was deprotected with hydrazine to afford the O-lactosyl glycopolypeptide pLac-sol or pLac-lipid, respectively.

A one-pot multi-enzyme system was used to α-2,6-sialylate common pLac precursors with N-acetyl neuraminic acid (for pSia), 9-N-propargylcarboxy N-acetylneuraminic acid (for pS7L), or N-propargylcarboxy mannosamine 4 with sodium pyruvate and a neuraminic acid aldolase (for pS9L, FIG. 2). After enzymatic elaboration, high-affinity Siglec ligands were synthesized by Huisgen cycloaddition using either adamantylazide (for pS7L) or benzhydrylazide (for pS9L). This afforded glycopolypeptides bearing either a C-terminal lipid or soluble group, a free N-terminus, and glycans bearing terminal high-affinity Siglec ligands. Finally, polypeptides were N-terminally labeled with commercially available biotin or AlexaFluor NHS esters (Methods).

### Example 2—pS9L-Lipid Inserts into Cell Membranes and Binds Siglec-9 in Cis

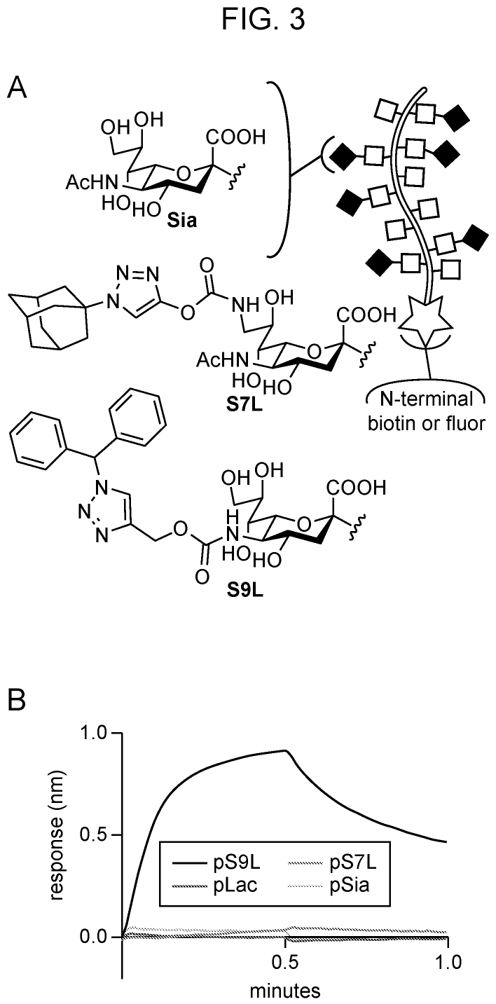

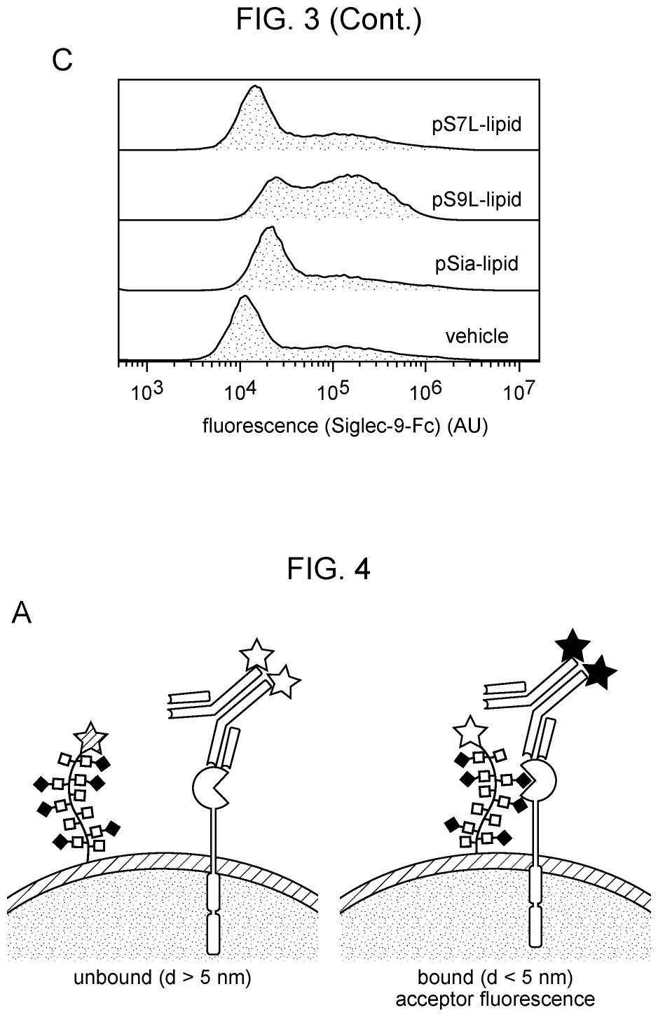

A panel of N-terminally labeled sialylated glycopolypeptides was constructed from the common precursors pLac-lipid or pLac-sol (FIG. 3, panel A). The binding of the constructs to recombinant soluble Siglec-Fc fusion proteins was tested in vitro and on cell surfaces. For in vitro binding, N-terminally biotinylated lipid-free glycopolypeptides were immobilized on streptavidin-coated tips and dipped into solutions of Siglec-Fc fusion proteins. Each glycopolypeptide bound specifically to its cognate Siglec receptor (FIG. 3, panel B). For example, pS9L, but not any other glycopolypeptide tested, bound to Siglec-9-Fc with high affinity (FIG. 3, panel B). Similar specificity was observed when lipid-linked versions were inserted into cell membranes and the cells were stained with recombinant Siglec-9-Fc (FIG. 3, panel C). There were no substantial differences between the insertion of various glycopolypeptides. Mutation of Siglec-9 R120A, a loss of sialic acid binding mutant, abrogated the effect observed in both the in vitro and flow experiments, and staining with SNA showed no increase in binding for any structures.

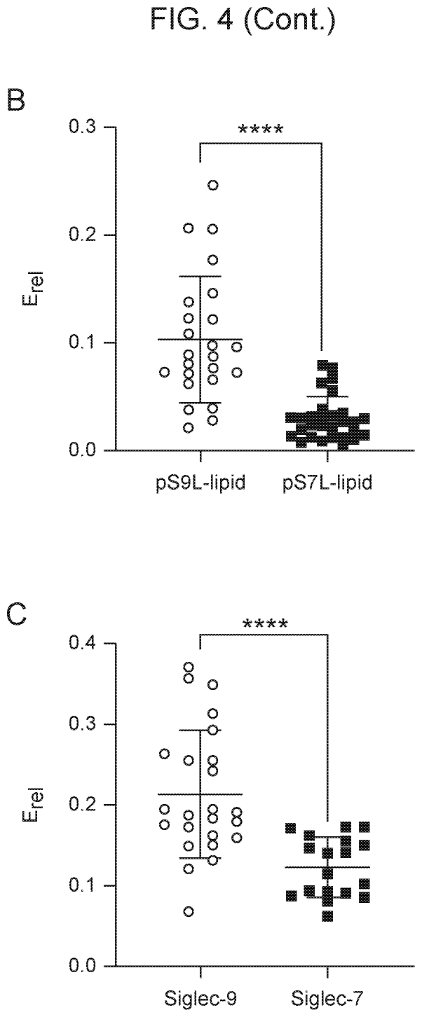

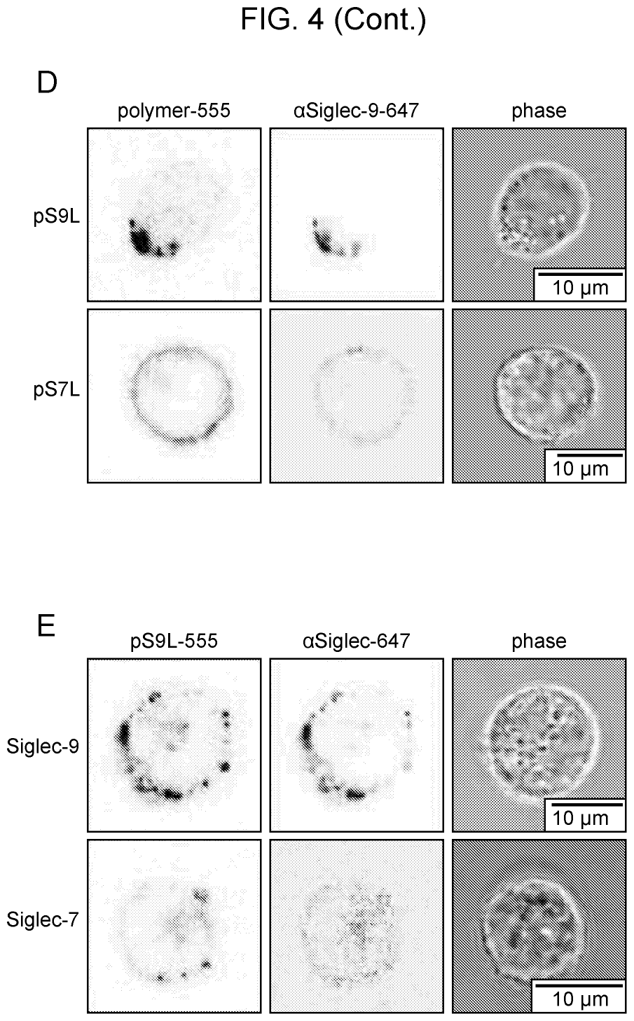

To determine whether lipid-tethered glycopolypeptides associate with Siglecs in cis, Förster Resonance Energy Transfer (FRET) between the fluorophores of lipid-linked glycopolypeptides N-terminally labeled with an AlexaFluor 555 donor fluorophore and anti-Siglec antibodies conjugated to an AlexaFluor 647 acceptor was measured (FIG. 4, panel A). The donor fluorophore was excited with a 535 nm laser and fluorescence was detected in both the 555 nm and 647 nm emission wavelengths. FRET signal was quantified using a simplified version of FRET efficiency known as relative efficiency (Eᵣₑₗ), which is the ratio of acceptor fluorescence intensity to the sum of acceptor and donor fluorescence intensities.

Glyocopolypeptide specificity was analyzed using pS9L-lipid and pS7L-lipid on Siglec-9 expressing cells (FIG. 4, panels B and D) and Siglec specificity using pS9L-lipid on Siglec-9 or -7 expressing cells (FIG. 4, panels C and E). Impressively, dramatic increases in relative FRET efficiency was observed only when pS9L-lipid was paired with Siglec-9, but not mismatched cases. Furthermore, intense nuncta in the Siglec-9/pS9L-lipid case were observed. Also determined was the intensity of acceptor fluorophore emission for the FRET case to the single color controls in order to account for differences in antibody binding affinities or antibody/fluorophore ratios. Observed was a substantial increase in acceptor emission intensity between FRET and acceptor-only controls of Siglec-9/pS9L-lipid, but not Siglec-7/pS9L-lipid.

### Example 3—Cis-Binding Qlycopolypeptides Inhibit TLR4-Induced NF-κB Activity in Siglec-9 Expressing Cells

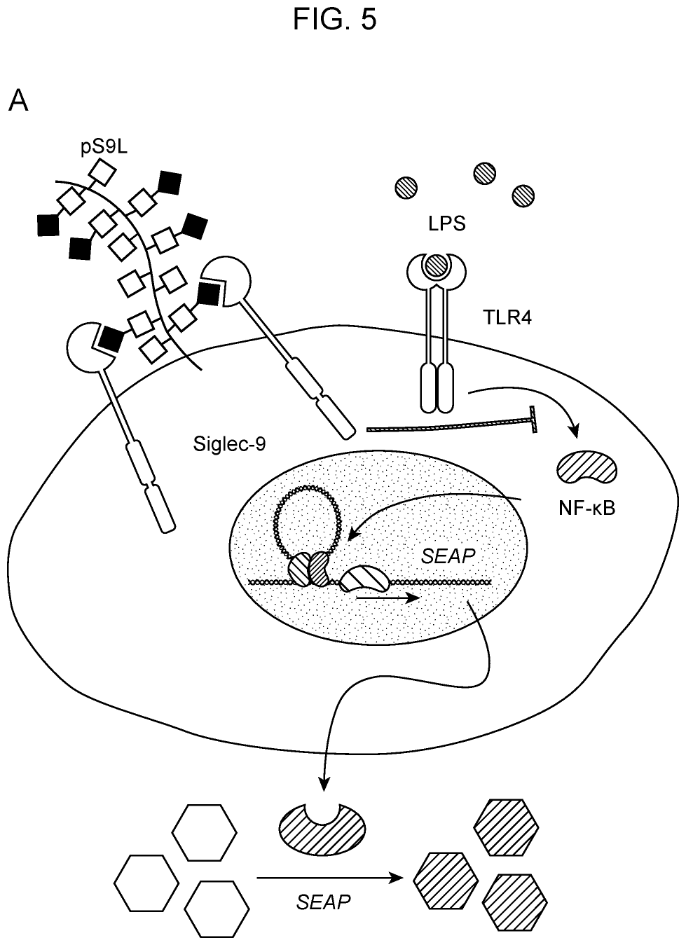

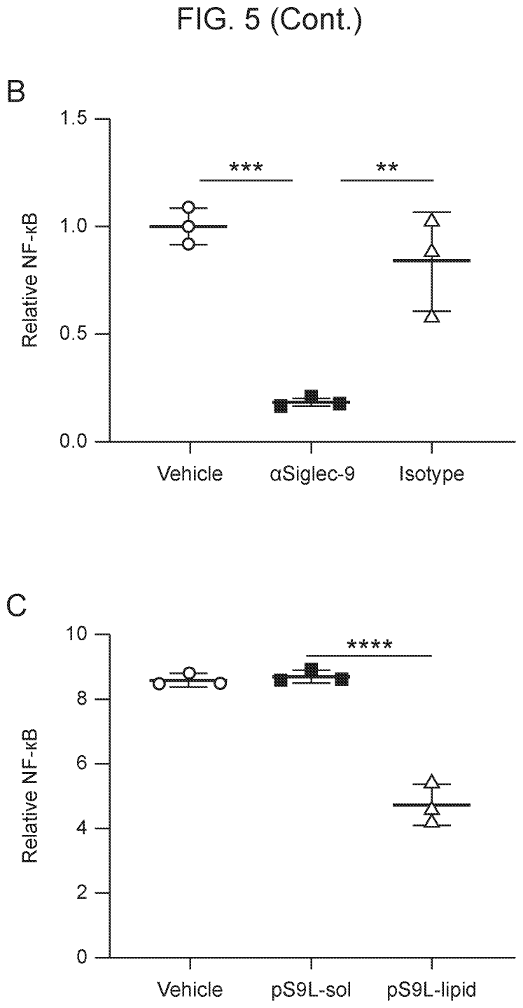

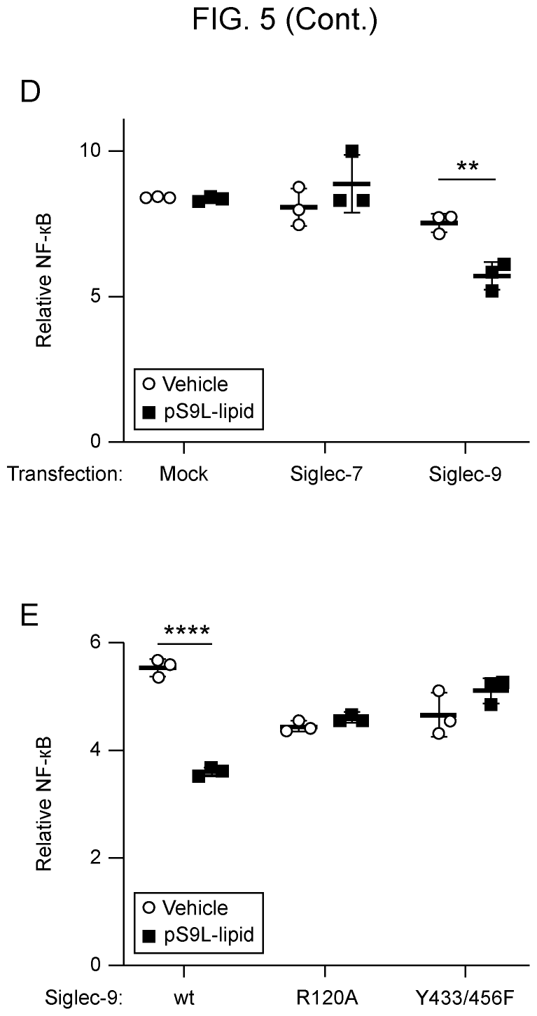

To examine the effect of membrane-tethered glycopolypeptides on inflammatory signaling, a reporter system for Siglec activity was developed based on the HEKBlue hTLR4 reporter assay. CD33-related Siglecs have been previously been shown to modulate hTLR4 in transgenic HEK cells. In this reporter line, LPS-induced TLR4 signaling initiates NF-1B transcription of an alkaline phosphatase (SEAP) that is secreted into the supernatant. NF-1B activity is correlated to SEAP activity in a colorimetric assay. This assay was modified by transfecting these cells with Siglec expression vectors (FIG. 5, panel A). The assay was validated by plating Siglec-9 expressing HEKBlue cells on anti-Siglec-9-coated plates to engage Siglec-9 signaling, and a substantial reduction in activity compared to vehicle-treated or isotype-coated plates was observed (FIG. 5, panel B).

HEKBlue cells were transfected with Siglec-9 and coated with glycopolypeptide (1 μM) before stimulation with LPS. Reduced relative NF-κB activity was observed with cis-binding pS9L-lipid, but not with the soluble trans-binding pS9L-sol (FIG. 5, panel C) or with other lipid-tethered glycopolypeptides.

To test the Siglec specificity of pS9L-lipid, HEKBlue cells were transfected with a Siglec-9, Siglec-7, or mock vector and coated with pS9L-lipid (1 μM) before stimulation with LPS. It was observed that pS9L-lipid only inhibited NF-κB activity compared to vehicle-treated cells when the cells express Siglec-9 (FIG. 5, panel D). Transfection of Siglec-9 constructs bearing mutation of R120A or a double mutant of Y433/456F, which prevents tyrosine phosphorylation of the ITIM/ISIM domains, rescued NF-1B activity in response to pS9L-lipid (FIG. 5, panel E).

### Example 4—Cis but not Trans Binding pS9L Inhibits MAPK Signaling in Macrophages

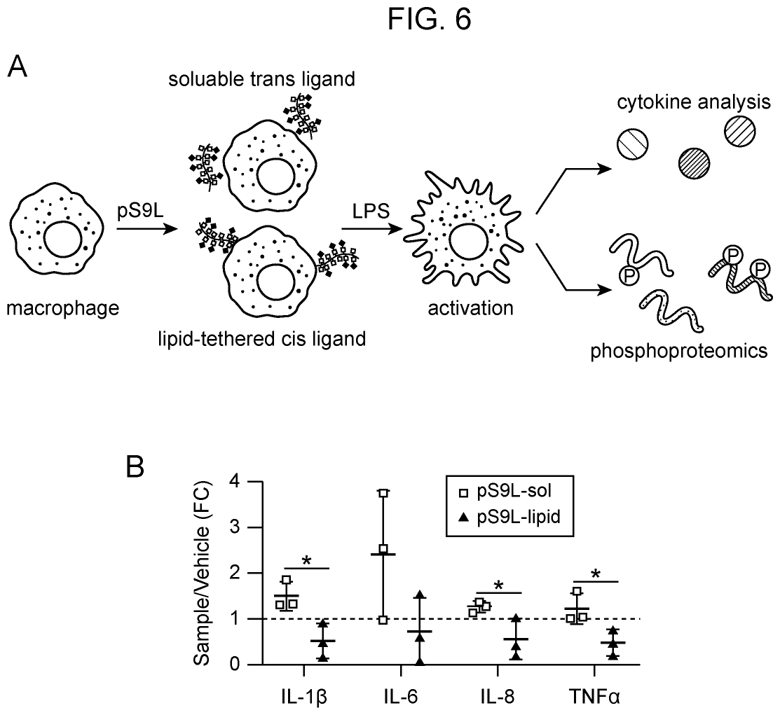

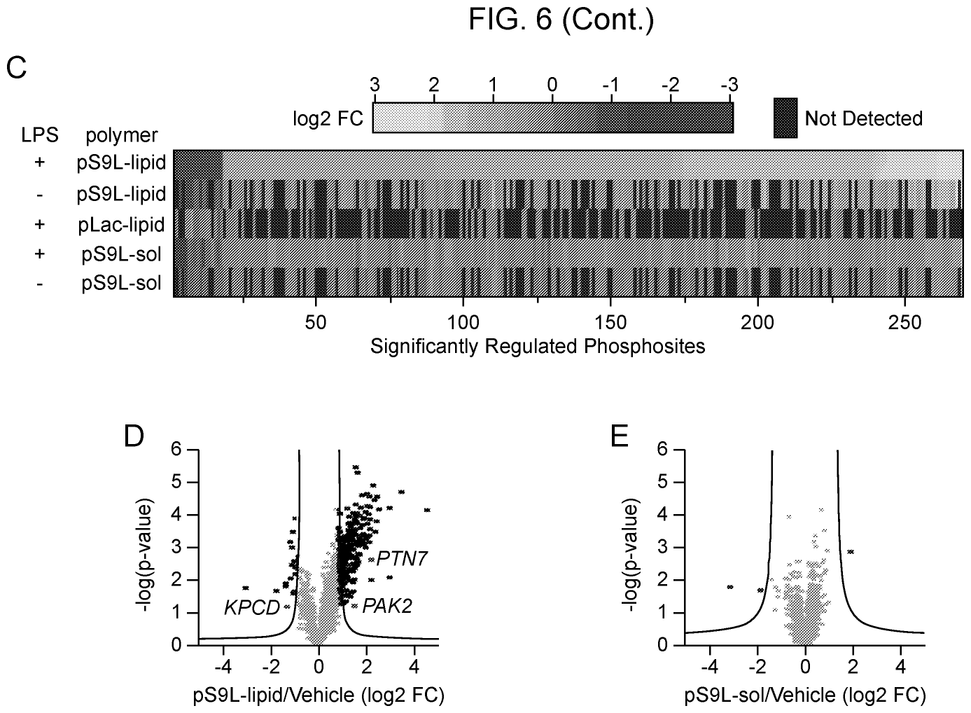

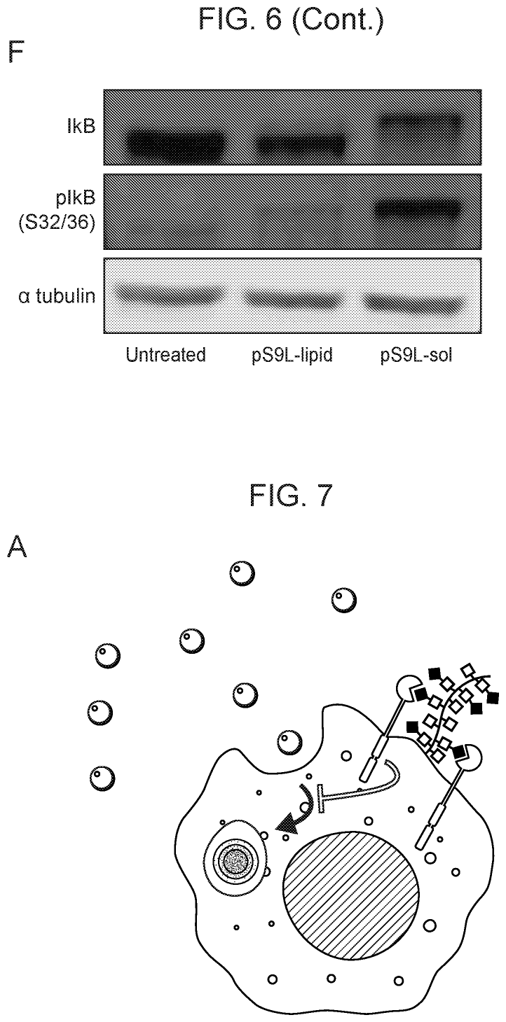

Pathologically relevant cell types expressing Siglec-9 in inflammatory disease are predominantly of the macrophage lineage. THP-1 cells are an immortalized monocyte line that are plastic and have been used to study macrophage biology. THP-1 monocytes were differentiated into Siglec-9⁺ macrophages using phorbol-12-myristate-13-acetate and these macrophages were used to interrogate the effect and mechanism of action of pS9L-lipid. Hyposialyl THP-1 macrophages were used as a model of hyperinflammatory macrophages, comparable to the hyposialylation previously used to potentiate activity that recapitulates the hyposialylation on hyperinflammatory phagocytes. Assessed was whether this model would deconvolve confounding effects of native cis ligands, permitting isolation of the pathway of pS9L-lipid signaling. The effects of the soluble trans-binding pS9L-sol and membrane-tethered cis-binding pS9L-lipid were compared to vehicle-treated cells by coating cells with glycopolypeptide and analyzing them with or without LPS stimulation. Early signaling cascades were analyzed using quantitative phosphoproteomics and this technique complemented with cytokine quantitation at a later timepoint (FIG. 6, panel A).

The cytokine production of cells was analyzed using a multiplexed cytometric bead assay for six inflammatory human cytokines. Macrophages were pretreated with glycopolypeptide (200 nM) followed by either vehicle or LPS stimulation for 18 h. Samples of the supernatant were taken and assayed for cytokine content. Marked decreases in IL-1β, IL-8, and TNFα was observed when treated with cis-binding pS9L-lipid but not the trans-binding soluble analogue pS9L-sol (FIG. 6, panel B). IL-10 and IL-12p70 were under the limit of detection for this assay (<20 pg/mL).

Using a similar protocol, changes in the phosphoproteome were analyzed from lysates either after glycopolypeptide loading or after LPS stimulation for 5 min (FIG. 6, panels C-E). Minimal changes were observed in unstimulated macrophages. Dramatic changes in phosphorylation were observed when LPS-challenged cells were pretreated with cis-binding pS9L-lipid, but not pS9L-sol or pLac-lipid. Notably, phosphorylation correlating to decreased activity of MAPK signaling was observed. Also observed was differential phosphorylation of SH2-domain containing proteins (e.g. SHIP2 and PTN7). Downstream MAPK signaling was validated by analysis of phosphorylation of total protein of IκB by Western blot (FIG. 6, panel F). It was found that pS9L-lipid had both more total IκB and less phosphorylation of IκB at sites (S32/36) that signal IκB degradation compared to pS9L-sol. Differential phosphorylation of phosphotyrosines on Siglec-9 was not observed at any timepoints assayed.

### Example 5—Cis-Ligands for Siglec-9 and -E Inhibit Phagocytosis by Macrophages and Microglia

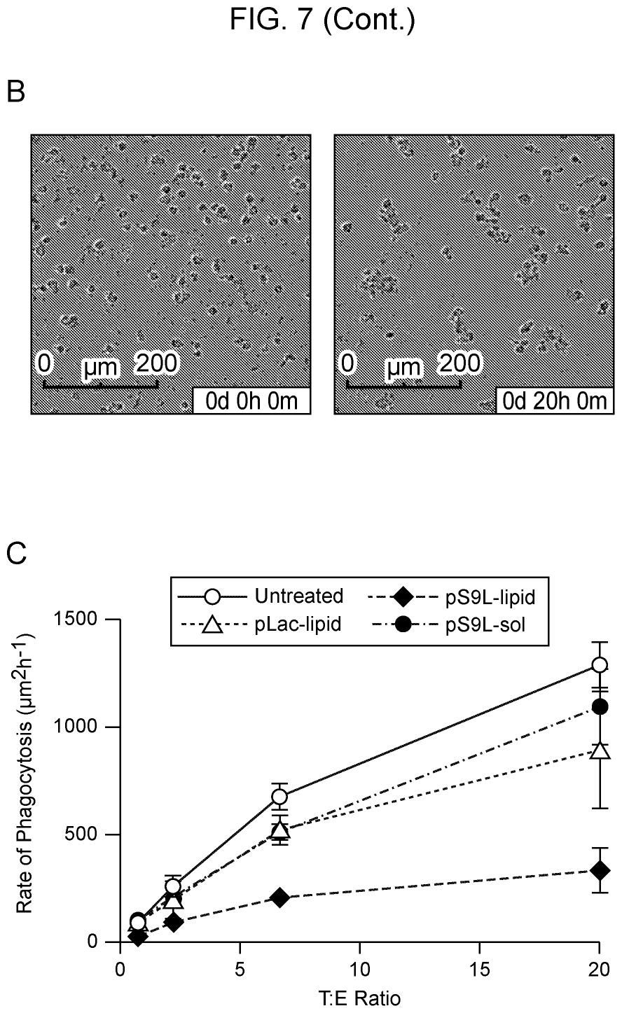

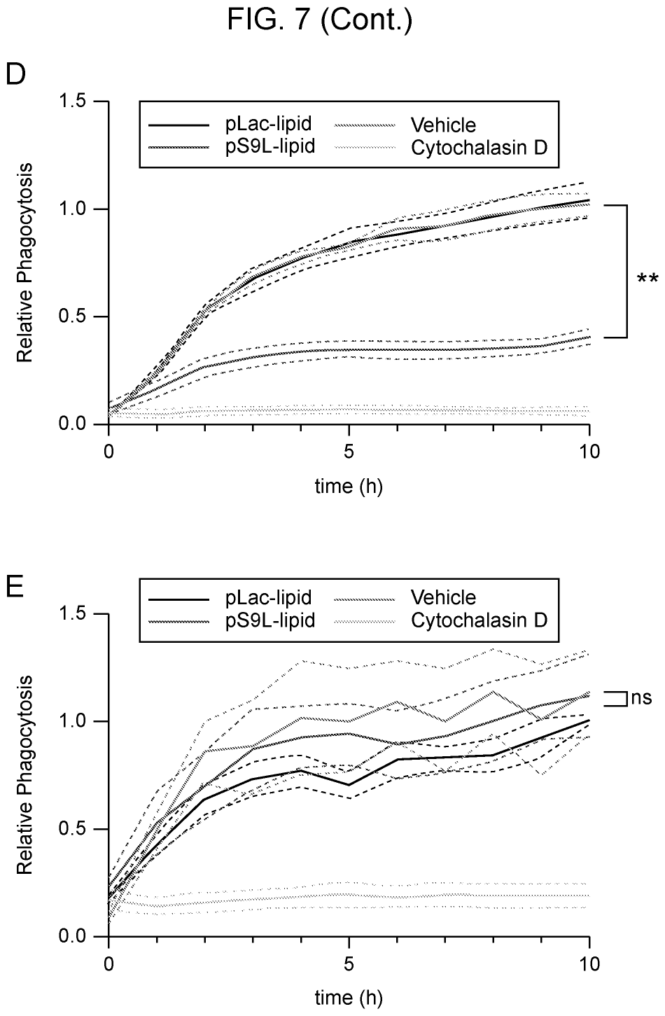

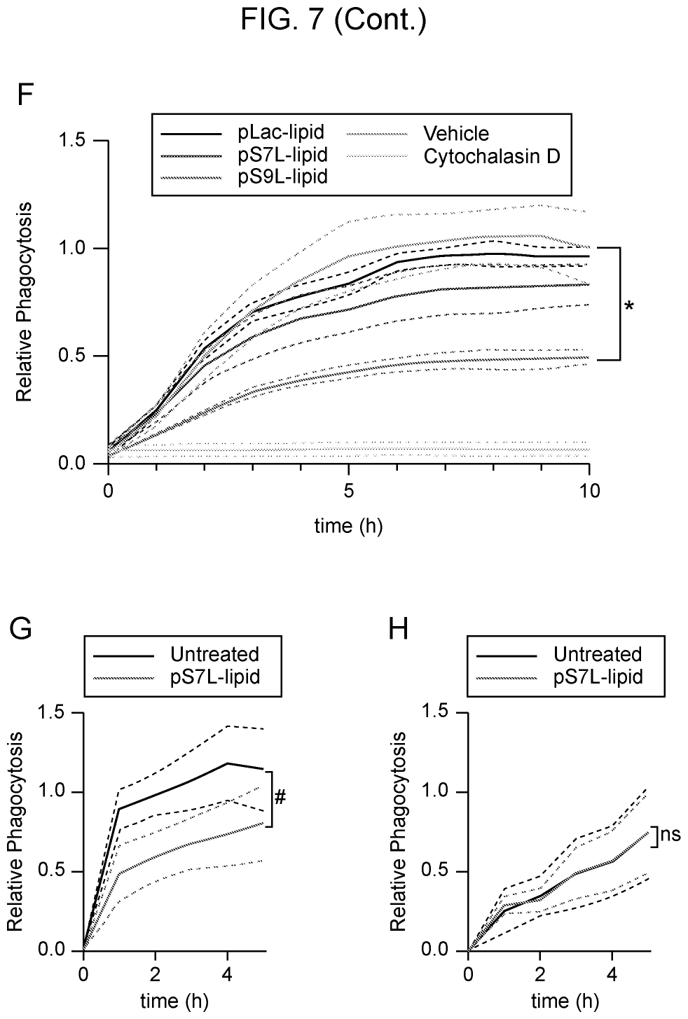

Engagement of Siglec receptors has been shown to inhibit phagocytosis. Assessed in this example was whether pS9L-lipid could inhibit phagocytosis via Siglec-9. This was studied by monitoring phagocytosis of low-pH turn-on fluorescent (pHrodo red) beads by microscopy (FIG. 7, panels A and B).

The initial rates of phagocytosis were analyzed at multiple effector to target (E:T) ratios (FIG. 7, panel C). Compared was pS9L-lipid to its a soluble analogue (pS9L-sol), a glycovariant bearing only inert lactose (pLac-lipid), and untreated cells to analyze any potential interactions of pS9L glycan binding or non-specific effects caused by lipid insertion. Glycopolypeptides were loaded onto wild-type THP-1 macrophages (200 nM) prior to the addition of varying amounts of target pHrodo-labeled beads. Phagocytosis was then monitored by microscopy immediately after addition of targets and then after 1 h to determine the initial rate of phagocytosis. Phagocytosis was quantified as the area of fluorescence above a background threshold observed over five images per well with three wells per sample. In the case of pS9L-lipid, we observed a dramatic reduction in the rate at any given E:T ratio and the apparent maximum velocity of phagocytosis, whereas both control glycopolypeptides yielded comparable results to vehicle treated cells.

To determine whether the effect observed was mediated by Siglec-9 agonism, two CRISPR knockouts were generated: one of Siglec-9 and one of CMAS, a gene necessary for sialic acid biosynthesis. KO of CMAS yields sialic-acid deficient macrophages, which was assessed to determine whether it would potentiate the effect. It was found that pS9L-lipid potently inhibited CMAS KO macrophages (FIG. 7, panel D), and that KO of Siglec-9 abrogated the effect of pS9L-lipid (FIG. 7, panel E).

Next, a small panel of glycovariants based on the same scaffold as pS9L-lipid was tested (FIG. 7, panel F). It was observed that only pS9L-lipid was able to significantly inhibit phagocytosis. A trend of inhibition by the Siglec-7-binding pS7L-lipid was observed, but it was not statistically significant; THP-1 macrophages express Siglec-7 at low levels. A panel of soluble trans binding glycopolypeptides bearing the same glycans and of similar molecular weight was also assayed but no effect on phagocytosis was observed. It was determined that inhibition by pS9L-lipid was dose-dependent on glycopolypeptide pretreatment and could be observed with alternate targets, including zymosan fungal particles.

To assess the generality of inhibition by clustering Siglecs with cis ligands, the mild inhibition by pS7L-lipid was followed up on. While the S7L sialoside has some affinity for Siglec-7, it was the strongest ligand found by Paulson and coworkers for Siglec-E, the murine orthologue of Siglecs-7/9. Thus, assessed was a potential inhibitory effect on Siglec-E expressing cells. Indeed, observed was a inhibitory trend in murine microglia pretreated with pS7L-lipid (FIG. 7, panel G) that was abrogated by CRISPR KO of Siglec-E (FIG. 7, panel H). A stronger and statistically significant effect was observed compared to a control polymer.

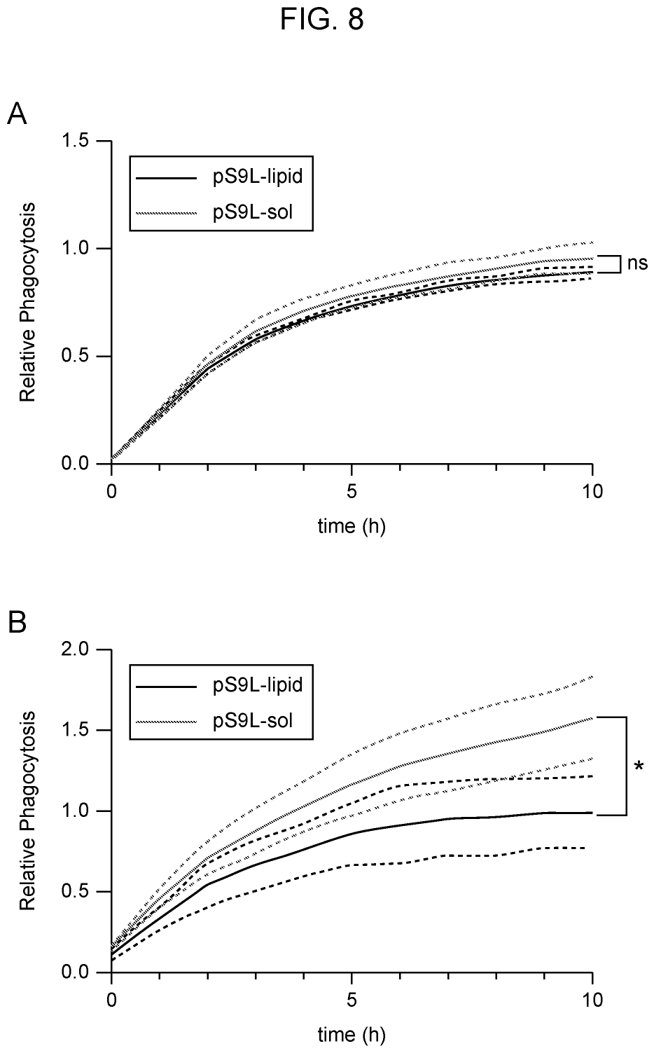

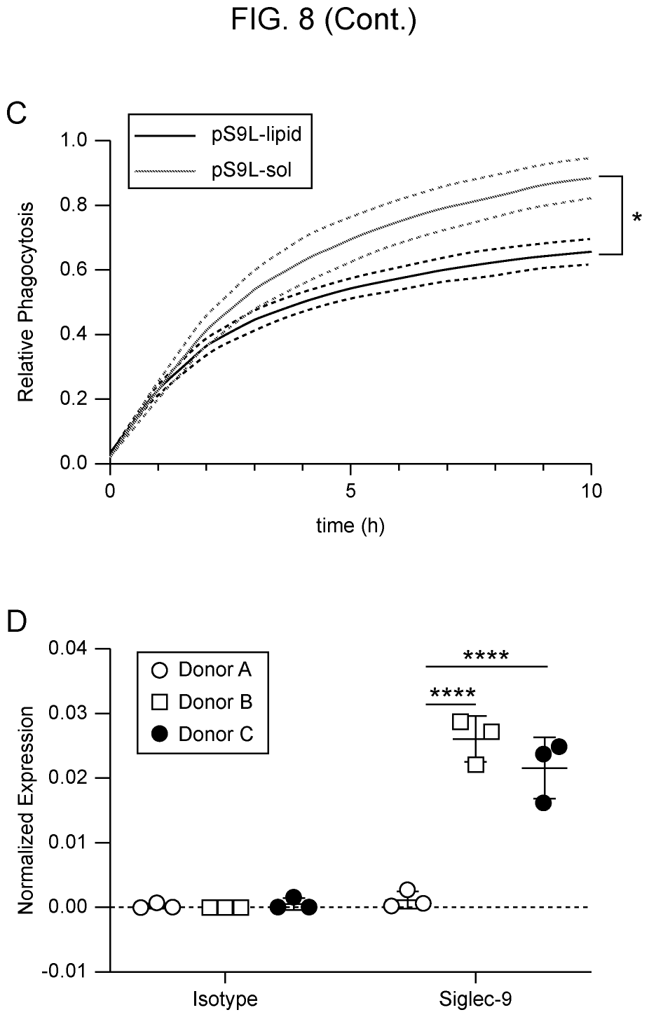

To demonstrate the clinical relevance of these findings, a similar assay was performed with primary human macrophages. Monocytes from healthy donor PBMCs were isolated and differentiated into resting (M0), M1, or M2 macrophages. When pretreated with glycopeptides, it was observed that the phagocytic activity of M0 and M1, but not M2 macrophages from five of six donors was inhibited by treatment with pS9L-lipid but not control polymers pS9L-sol or pLac-lipid (FIG. 8, panels A-C). Following up on the single non-responsive donor, it was determined that macrophages from this donor had dramatically lower levels of Siglec-9 expression (FIG. 8, panel D).

### Methods for Examples 1-5

Statistical Analysis

All statistical analyses were performed using GraphPad Prism 6.

Glycopolypeptide Synthesis

Glycopolypeptides were synthesized as previously described (Delaveris et al. (2019) Membrane-Tethered Mucin-like Polypeptides Sterically Inhibit Binding and Slow Fusion Kinetics of Influenza A Virus. ChemRxiv). In brief, N-carboxyanhydrides of alanine and O-lactosylserine were polymerized using precomplexed initiators to afford lipid-linked or soluble protected glycopolypeptides. The glycans were deacetylated using hydrazine and purified by dialysis. The polylactosyl scaffolds were then elaborated using a one-pot multi-enzyme system to afford various sialosides on the glycopolypeptide scaffold. Unnatural sialosides bearing alkyne handles were then reacted with azides to afford a polymeric presentation of previously described high-affinity Siglec ligands.

Human Cell Culture

Cell lines were cultured in either DMEM (HEKBlue hTLR4, BV2) or RPMI (JURKAT, THP-1) supplemented with 10% heat-inactivated FBS. THP-1 cells were further supplemented with 50 μM betamercaptoethanol. THP-1 monocytes were differentiated into macrophages by activating with PMA for 24 h followed by recovering in normal media for 24 h. PBMCs were isolated from buffy coats from whole blood or LRS chambers using Ficoll-Paque gradient centrifugation. Monocytes were isolated by adherence onto tissue culture plastic. and monocytes were differentiated into macrophages in RPMI-1640 containing 20% heat-inactivated FBS for 7 d with either no exogenous cytokines (M0), GM-CSF (immature M1), GM-CSF for 5 d followed by LPS and IFN-γ in 10% heat-inactivated FBS for 2 d (activated M1), or M-CSF for 5 d followed by IL-4 and IL-13 in 10% heat-inactivated FBS for 2 d (M2).

In Vitro Protein Binding

Protein binding was recorded on an OctetRed96 using biotinylated ligands (200 nM) in PBS with BSA (0.1%) loaded onto streptavidin-coated tips for 60 s (˜0.4 nm response). Tips were then dipped into a serial dilution of Siglec-Fc and associated for 30 s and then dissociated in buffer for 30 s. Tips were regenerated between washes in pH 1.5 glycine buffer.

Flow Cytometry

Cells were harvested and loaded with fluorophore-conjugated glycopolypeptide in serum-free media at a density of 10⁷ cells/mL for 1 h with gentle agitation every 15 min. The cells were then washed and stained with either a fluorophore-conjugated primary antibody or an unconjugated primary antibody with a fluorophore-conjugated anti-IgG secondary antibody at 4° C. and washed three times after staining. All flow analysis was done on unfixed cells.

Fluorescence Microscopy

FRET data were collected on a confocal microscope. JURKATs expressing Siglec-7 or -9 were suspended in serum-free RPMI 10⁷ cells/mL and labeled with AlexaFluor555-labeled glycopolypeptide (2 μM) for 1 h with gentle agitation every 15 min. Cells were washed and then labeled with AlexaFluor647-labeled anti-Siglec-7 or -9 antibody for 30 min at room temperature in complete media. The cells were washed with PBS and then plated onto live-cell imaging glass 8-well borosilicate #1.5 cover slips precoated with fibronectin. Cells were then imaged. For Siglec-9 immunocytochemistry, cells were fixed in 10% formalin, washed, and stained with AlexaFluor488-conjugated anti-Siglec-9 for 1 h on ice. Cells were then washed and imaged by 488 nm fluorescence on an Incucyte collecting five images per well with three wells per condition.

Cloning

An expression plasmid for PmNanA was constructed by InFusion cloning from a gBlock from IDT ligated into a PCR-linearized pET22b vector. CRISPR plasmids were constructed using optimized guides and cloned into the LentiCRISPR v2 plasmids using the Gecko protocols and purified by MiraPrep. pCMV Siglec-9 mutants were generated using a Q5 mutagenesis kit.

Protein Expression and Purification

PmNanA, Pd26ST, and NmCSS were expressed in BL21(DE3) E. coli and isolated.

HEKBlue hTLR4 Reporter Assay

The HEKBlue hTLR4 assay was generally performed according to manufacturer's instructions Cells were transfected 24 h before the assay using Lipofectamine LTX. For antibody-coated plate assays, plates were prepared by incubating 96-well plates with solutions antibody (10 ng/mL) in PBS for 2 h at 37° C. and then washed three times with PBS before plating transfected cells. For glycopolypeptide assays, cells were harvested from the transfection plates, pelleted by centrifugation (300 rcf, 5 min), and resuspended in a solution of glycopolypeptide (1 μM) in serum-free DMEM. Cells were mixed every 15 min for 1 h, at which point cells were washed with 1 mL complete media, counted, and plated.

Cytokine Bead Assay

CMAS KO THP-1 macrophages were cultured and labeled with glycopolypeptide (200 nM) for 3 h. At this point, media and either vehicle or LPS (100 μg/mL) were added and cells were cultured for 18 h. Aliquots of media were then taken and flash frozen at −80° C. The BD human inflammatory cytokine bead quantitation was then performed on thawed samples from three biological replicates in one batch, according to manufacturer's instructions.

Phosphoproteomics

CMAS KO THP-1 macrophages were cultured and labeled with glycopolypeptide (500 nM) in serum free media for 3 h. At this point, media and either vehicle or LPS (100 μg/mL) were added and cells were stimulated for 5 min. Cells were then lysed in cold RIPA buffer with benzonase, pelleted by centrifugation (18000 rcf, 15 min, 4° C.), and supernatant protein concentrations were quantitated by Rapid Gold BCA.

Proteins were digested into tryptic peptides using an S-trap protocol (Protifi) and were subsequently labeled with 10-plex TMT (Tandem Mass Tags, Thermo Fisher Scientific). Phosphopeptides were enriched with Ti(IV)-IMAC beads (ReSyn Biosciences). Phosphopeptides and protein abundance samples were analyzed by LC-MS/MS using a Dionex Ultimate 3000 RPLC nano system coupled to an Orbitrap Fusion (Thermo Fisher Scientific). Peptides were loaded on to a trap column (Acclaim PepMap 100 C18, 5 um particles, 20 mm length, Thermo Fisher Scientific) and separated over a 25 cm EasySpray reversed phase LC column (75 μm inner diameter packed with 2 μm, 100 Å, PepMap C18 particles, Thermo Fisher Scientific) using water with 0.2% formic acid (mobile phase A) and acetonitrile with 0.2% formic acid (mobile phase B). All methods totaled 180 minutes of acquisition time per analysis. Raw files were searched using the Andromeda algorithm and processed in MaxQuant. Results were then processed in Perseus to calculate statistically significant changes in the phosphoproteome. Data have been deposited to the ProteomeXchange Consortium via the PRIDE partner repository with the dataset identifier PXD018774.

Western Blot

CMAS KO THP-1 macrophages were cultured and labeled with glycopolypeptide (500 nM) in serum free media for 3 h. At this point, media and either vehicle or LPS (100 μg/mL) were added and cells were stimulated for 60 min. Cells were then lysed in cold RIPA buffer with benzonase, pelleted by centrifugation (18000 rcf, 15 min, 4° C.), and supernatant protein concentrations were quantitated by BCA. Lysates were then run on SDS-PAGE using a 4-12% bisacrylamide gel at 200 V for 1 h in XT-MES. The gel was transferred to nitrocellulose using a TransBlot Turbo using the standard TurboBlot conditions. The blot was blocked with 5% BSA in TBS and stained with primary antibodies overnight at 4° C., followed by incubation with an IR-dye labeled secondary antibody at room temperature for 1 h. Blots were imaged by LiCOR.

Phagocytosis Assays

Phagocytes were treated with glycopolypeptide for 3 h in serum free media. The cells were washed and coated with 100 μL serum free media. Targets were then added as a suspension in 100 μL serum free media. The plates were briefly centrifuged (300 rcf, 1 min) to settle the targets, and then phagocytosis was monitored by fluorescence microscopy on an Incucyte. Five images were collected per well for three wells per condition. For BV2 phagocytosis, BV2 cells were pretreated with recombinant, endotoxin-free V. cholera sialidase for 1 h at 2 μM prior to treating with glycopolypeptides.

### Example 6—Cis-Binding Siglec Agonists Inhibit Neutrophil Activation

TLR-7/8 Agonist R848 Induces NETosis of Primary Neutrophils In Vitro

Neutrophils are immune cells of the myeloid lineage that are involved in numerous innate immune functions. It has been suggested that neutrophils drive a hyperinflammatory response in COVID-19 through a death process called NETosis, in which neutrophils rapidly decondense chromatin and spew out a neutrophil extracellular trap (NET), an amalgam of genomic DNA, intracellular proteins (e.g., histones), and tissue-damaging enzymes (e.g., neutrophil elastase, myeloperoxidase). Extracellular DNA and tissue damage from NET-associated enzymes act as proinflammatory signals to other immune cells and are proposed to initiate the hyperinflammatory cascade in COVID-19, leading to ARDS and potentially death. Consistent with this hypothesis, NETs have been extensively observed both at the site of infection (i.e., pulmonary tissue) and in the periphery (i.e., sera and plasma).

In COVID-19, evidence of extensive NETosis can be observed in infected lungs, and SARS-CoV-2 virions have been shown to infect and induce NETosis of healthy neutrophils in vitro. These reports implicate TLR-7 and/or TLR-8 in inducing NETosis of neutrophils at the site of infection. Notably, TLR-7 and TLR-8 are single-stranded RNA receptors with numerous substrates identified in the SARS-CoV-2 genome. Consistent with the hypothesis that SARS-CoV-2 induces TLR-7/8-mediated immunity, human genetic variations in TLR7 are associated with severe COVID-19. Thus, agonists of TLR-7/8 may provide a convenient means of modeling local inflammation induced by viral infection in vitro without using live virus.

TLR agonists were assayed using the live-cell imaging techniques in which freshly isolated neutrophils are cultured in low-serum media in the presence of a fluorogenic and membrane impermeable DNA-intercalating dye (Cytotox Green). Upon genomic DNA-externalization by NETosis, dye intercalates and fluorescence increases. As previously demonstrated, because NETs are much larger than the nuclei of apoptotic cells, NETotic cells yield much larger areas of fluorescence than apoptotic cells, as observed by microscopy. Thus, apoptotic cells can be filtered out by only counting large (i.e., >>100 μm²) fluorescent objects.

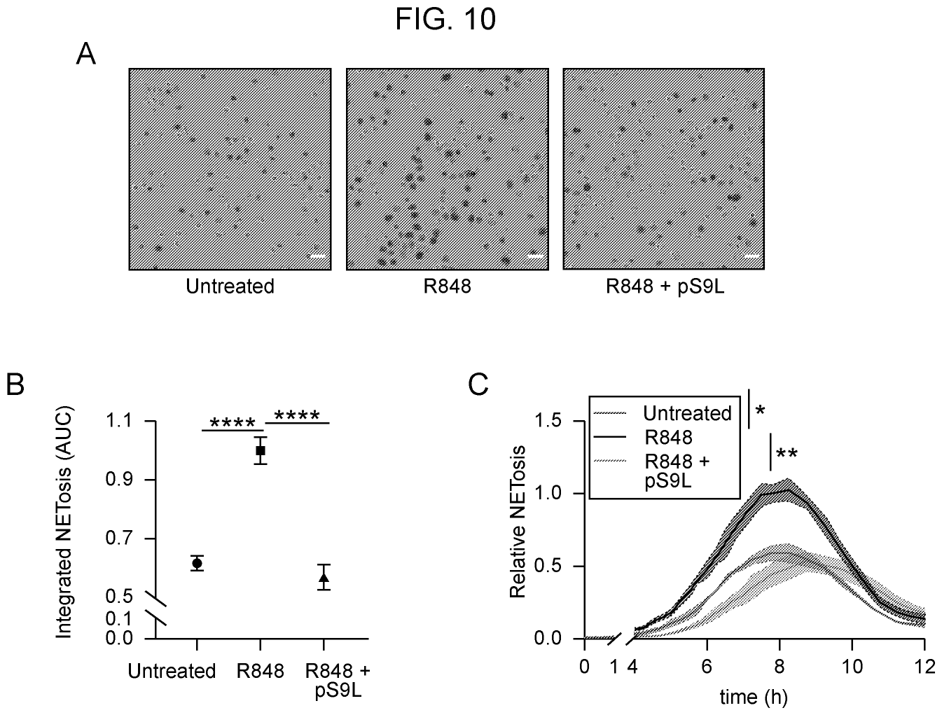

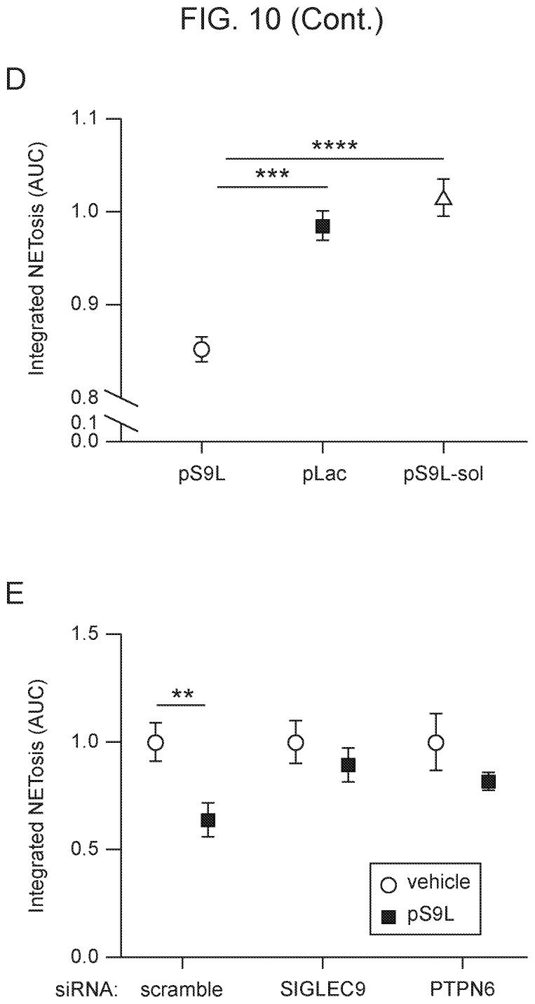

In the present studies, it was found that a TLR-7/8 agonist, R848, was sufficient to induce NETosis of healthy neutrophils in vitro (FIG. 10a-c). The citrullination status of the PADI4 substrate H3 was also assayed by Western blot, and it was observed that R848 rapidly induced citrullination at R2, R8, and R17. While citrullination is an important aspect of NETosis, the extent of citrullination is not necessarily indicative of the extent of NETosis as, for example, PMA-induced NETosis only yields moderate citrullination (data not shown). Additionally, performed was quantitative phosphoproteomics with lysates of neutrophils treated with media, phorbol-12-myristate-13-acetate (PMA), or R848. Similar results to previously published datasets using neutrophils stimulated with either R848 or PMA was observed. Furthermore, several phosphosites were found to be differentially regulated in both datasets, including those involved in neutrophil degranulation and calcium flux, consistent with the described mechanism of NETotic cell death. These results indicate that the TLR-7/8 agonist R848 induces NETosis in primary neutrophils. Thus, this compound can be used to model local inflammation associated with viral infection, including in COVID-19.

A Siglec-9 Agonist Inhibits TLR-7/8-Induced NETosis Via SHP-1

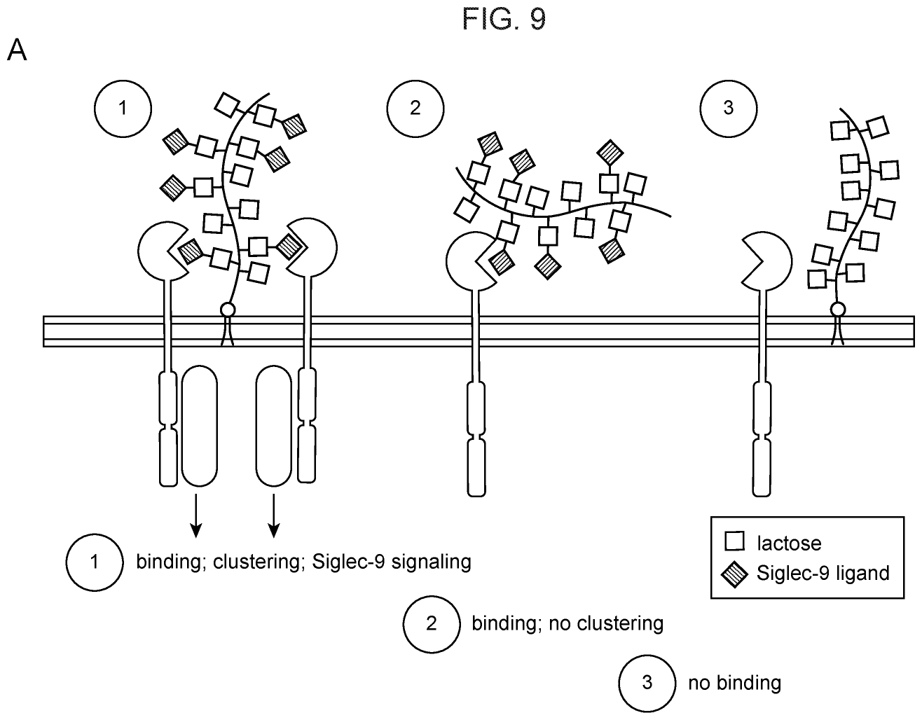

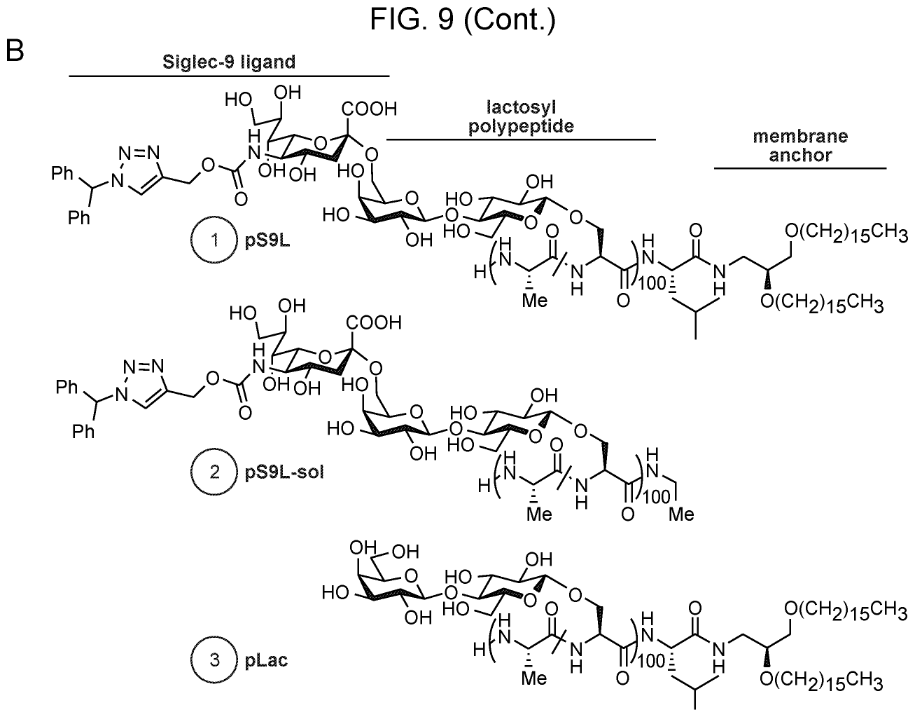

Previous work has shown that engagement of Siglec-9 leads to apoptotic and nonapoptotic death pathways as well as immunosuppression in neutrophils. Thus, it was hypothesized that Siglec-9 mediated immunosuppression and cell death could override the NETotic effect of antiviral TLR signaling. To test this notion, the Siglec-9 agonist pS9L was used, as well as the two control glycopolypeptides pLac and pS9L-sol (FIG. 9). Anti-NETotic activity was assayed by cotreatment of glycopolypeptide (500 nM) with R848 (10 μM) in primary neutrophils in the live-cell assay described above (FIG. 10). It was observed that pS9L was sufficient to inhibit NETosis induced by R848 treatment (FIG. 10a-c). Moreover, neither control polymer inhibited R848-induced NETosis (FIG. 10d). Also confirmed was that pS9L inhibits NETosis comparably to high concentrations of crosslinked anti-Siglec-9 antibody (clone 191240). The generation of mitochondrial-derived reactive oxygen species (ROS) was previously described as an important signaling step of Siglec-9-induced apoptotic signaling. Found here was that treatment with pS9L, in the absence of any TLR ligand so as to avoid NADPH-derived ROS in inflammatory signaling, induced an oxidative burst, as did treatment with a crosslinked anti-Siglec-9 antibody. Furthermore, the oxidative burst was inhibited by the addition of the SHP-1/2 inhibitor NSC-87877, suggesting that SHP-1 and/or SHP-2 mediate pS9L-induced oxidative burst in neutrophils, consistent with Siglec-9 engagement.

Quantitative phosphoproteomics was performed using lysates of R848-stimulated primary neutrophils cotreated with vehicle, pS9L, or pLac. Notably observed was increased phosphorylation of hyccin (HYCCI/FAM126A), a key component in phosphorylation of phosphoinositides, a class of signaling molecules implicated in mediating NETosis. Additionally observed was increased phosphorylation of RASAL3 (RASL3), a negative regulator of the MAPK signaling pathway. These data suggest that pS9L inhibits the calcium flux and NADPH activity necessary for NETosis, as well as the MAPK-suppressive effects that have been previously described for pS9L in macrophages.

To determine whether the anti-NETotic effect of pS9L is specifically mediated by Siglec-9 signaling, these results were recapitulated in the promyelocytic leukemia cell line HL-60. These cells can be differentiated into a neutrophil-like cells (dHL-60) using all-trans retinoic acid (ATRA, 100 nM) and dimethylsulfoxide (DMSO, 1.25% v/v). Notably, dHL-60 cells have previously been used to study NETosis in vitro. Consistent with prior reports, R848 induced NETosis in dHL-60 cells. Further observed was that pS9L inhibited NETosis and that siRNA knockdown of Siglec-9 (encoded by SIGLEC9) or SHP-1 (encoded by PTPN6) abrogated the effect of pS9L (FIG. 10e). Therefore, the Siglec-9 agonist pS9L inhibits TLR7/8-induced NETosis via Siglec-9 and SHP-1.

Siglec-9 is Upregulated in Severe COVID-19 and can Suppress NETosis Induced by COVID-19 Plasma

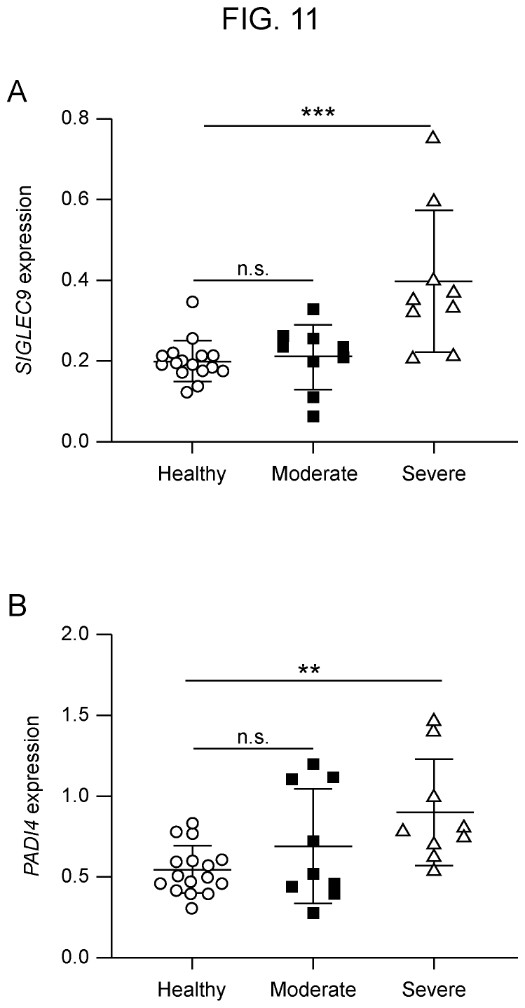

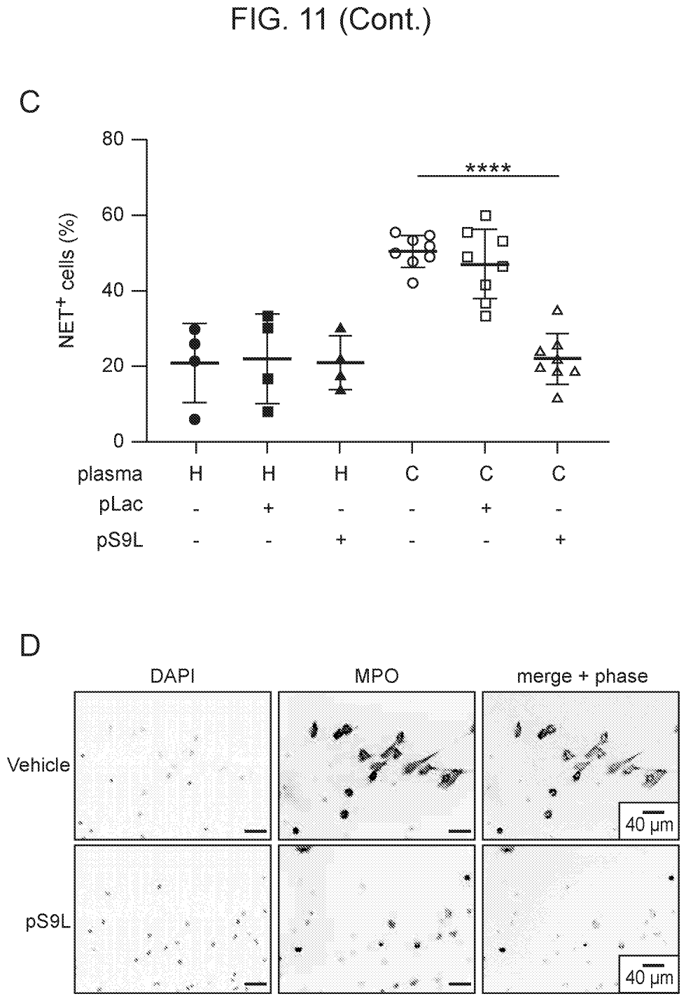

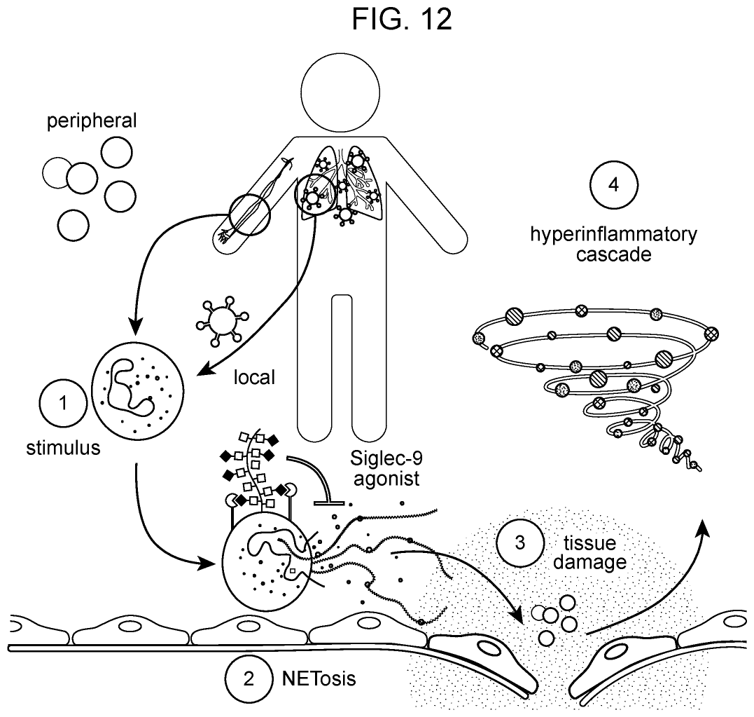

Sera and plasma from COVID-19 patients have been shown to induce NETosis of neutrophils isolated from healthy donors in vitro. The causative components are unclear, however potential factors include viral TLR ligands, damage-associated molecular patterns that bind TLRs, activated platelets, and (pro)inflammatory cytokines. Recent reports have described increased levels of neutrophil-activating cytokines in COVID-19 plasma, predominantly IL-8 and G-CSF. That the combination of IL-8 and G-CSF was sufficient to induce NETosis in vitro was also observed in the present studies. Additionally, transcriptomic analyses of peripheral myeloid cells and neutrophils in COVID-19 patients have revealed increased SIGLEC9 expression (FIG. 11a) and PADI4 expression (FIG. 11b). It is hypothesized that this is an exhaustion-like phenotype in which Siglec-9 expression is induced on hyper-NETotic neutrophils, similar to what has been observed with Siglec-9 on exhausted tumor-infiltrating T cells. These observations further support Siglec-9 an attractive target for therapeutic blockade of hyperinflammatory NETosis generally and in COVID-19 in particular.

To test the hypothesis that pS9L can inhibit NETosis induced by COVID-19 plasma, neutrophils isolated from whole blood of healthy donors were treated with citrate-anticoagulated heterologous plasma from healthy donors or COVID-19 patients. Neutrophils in undiluted plasma were cotreated with pS9L (500 nM), the non-binding analog pLac (500 nM), or vehicle. To satisfy biosafety restrictions, cells were incubated in the presence of COVID-19 plasma for 4 h and then fixed before assaying for extracellular complexes of myeloperoxidase (MPO) and DNA (DAPI) (FIG. 11c,d). The combination of these stains, which when observed extracellularly is indicative of NETosis, has been previously used to identified NET⁺ cells in the context of COVID-19. In the present studies, it was observed that COVID-19 plasma induced NETosis of neutrophils from healthy donors, as indicated by the formation of web-like NET structures (FIG. 11d). As in previous experiments with R848, COVID-19 plasma-stimulated NETosis was inhibited by pS9L treatment (FIG. 11c,d). Furthermore, pLac did not inhibit NETosis induced by COVID-19 plasma, and neither pS9L nor pLac affected basal NETosis of in vitro cultured neutrophils (FIG. 11c). Similar experiments were performed staining neutrophils treated with 10% plasma in IMDM or undiluted plasma for extracellular H1/DNA complexes, another marker of NETs, and observed comparable results.

Collectively, these data demonstrate that Siglec-9 agonism inhibits NETosis induced by COVID-19 patient plasma, and thus could inhibit peripheral inflammation in patients with COVID-19. Additionally, Siglec-9 agonists could resolve NET-associated pathologies generally including those observed in COVID-19 and elsewhere such as immunothrombosis and sepsis.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A cis-binding Siglec agonist comprising:
a scaffold bearing Siglec ligands; and
a membrane-tethering domain.

2. The Siglec agonist of claim 1, wherein the scaffold bearing Siglec ligands comprises a polymer scaffold.

3. The Siglec agonist of claim 2, wherein the scaffold bearing Siglec ligands comprises a glycopolypeptide scaffold.

4. The Siglec agonist of any one of claims 1 to 3, wherein the scaffold comprises from 2 to 50 Siglec ligands.

5. The Siglec agonist of claim 4, wherein the scaffold comprises from 2 to 10 Siglec ligands.

6. The Siglec agonist of any one of claims 1 to 5, wherein the Siglec ligands comprise immunosuppressive Siglec ligands.

7. The Siglec agonist of claim 6, wherein the Siglec ligands comprise ligands for one or more CD33-related Siglecs.

8. The Siglec agonist of claim 7, wherein the Siglec ligands comprise Siglec-9 ligands.

9. The Siglec agonist of claim 8, wherein the Siglec ligands exclusively comprise Siglec-9 ligands.

10. The Siglec agonist of claim 7, wherein the Siglec ligands comprise Siglec-7 ligands.

11. The Siglec agonist of claim 10, wherein the Siglec ligands exclusively comprise Siglec-7 ligands.

12. The Siglec agonist of any one of claims 1 to 5, wherein the membrane-tethering domain comprises a lipid membrane-tethering domain.

13. A composition comprising the Siglec agonist of any one of claims 1 to 12 present in a liquid medium.

14. A composition comprising the Siglec agonist of any one of claims 1 to 12 present in lyophilized form.

15. A pharmaceutical composition comprising:
the Siglec agonist of any one of claims 1 to 12; and
a pharmaceutically acceptable carrier.

16. The pharmaceutical composition of claim 15, wherein the composition is formulated for parenteral administration.

17. The pharmaceutical composition of claim 16, wherein the composition is formulated for intravenous administration.

18. A method of agonizing Siglec activity, the method comprising contacting a cell expressing Siglecs with the Siglec agonist of any one of claims 1 to 12 under conditions in which the membrane-tethering domain inserts into the cell membrane and the Siglec ligands bind in cis to one or more Siglecs expressed by the cell.

19. The method according to claim 18, wherein the method is performed in vitro.

20. The method according to claim 18, wherein the method is performed in vivo.

21. The method according to any one of claims 18 to 20, which is a method of agonizing Siglec-9 activity, wherein the Siglec agonist comprises Siglec-9 ligands.

22. A method of agonizing Siglec activity in an individual in need thereof, the method comprising administering to the individual an effective amount of the Siglec agonist of any one of claims 1 to 12.

23. The method according to claim 22, wherein the individual is in need of suppression of immune cell reactivity and the Siglec ligands comprise immunosuppressive Siglec ligands.

24. The method according to claim 22 or claim 23, wherein the individual has an inflammatory disease and the Siglec agonist is administered to the individual in an amount effective to treat the inflammatory disease.

25. The method according to claim 24, wherein the individual has an inflammatory disease selected from the group consisting of: age related macular degeneration, neutrophilic acute respiratory distress syndrome, systemic lupus erythematosus (SLE), eosinophilic gastroenteritis, allergy, asthma, autoimmune disease, coeliac disease, glomerulonephritis, hepatitis, inflammatory bowel disease, preperfusion injury, transplant rejection, and any combination thereof.

26. The method according to claim 22 or claim 23, wherein the individual has a viral infection.

27. The method according to claim 26, wherein the viral infection is a coronavirus infection.

28. The method according to claim 27, wherein the coronavirus infection is a SARS-CoV-2 infection.

29. The method according to any one of claims 22 to 28, wherein the Siglec agonist inhibits neutrophil activation in the individual.

30. The method according to any one of claims 22 to 29, wherein the Siglec agonist inhibits NETosis in the individual.

31. The method according to any one of claims 22 to 30, wherein the Siglec ligands comprise ligands for one or more CD33-related Siglecs.

32. The method according to claim 31, wherein the Siglec ligands comprise Siglec-9 ligands.

33. The method according to claim 32, wherein the Siglec ligands exclusively comprise Siglec-9 ligands.

34. The method according to claim 31, wherein the Siglec ligands comprise Siglec-7 ligands.

35. The method according to claim 34, wherein the Siglec ligands exclusively comprise Siglec-7 ligands.

36. A kit, comprising:
the pharmaceutical composition of any one of claims 15 to 17; and
instructions for administering an effective amount of the pharmaceutical composition to an individual in need thereof.

37. The kit according to claim 36, wherein the Siglec ligands comprise immunosuppressive Siglec ligands.

38. The kit according to claim 37, wherein the instructions are for administering an effective amount of the pharmaceutical composition to an individual in need of suppression of immune cell reactivity.

39. A method of making a cis-binding Siglec agonist, comprising:
synthesizing a polymer scaffold comprising a membrane-tethering domain at a terminus thereof; and
attaching Siglec ligands to subunits of the polymer scaffold.

40. The method according to claim 39, wherein the attaching comprises sialylating subunits of the polymer scaffold.
